(19) United States
(12) Patent Application Publication
(10) Pub. No.: US 2010/0152140 A1
(43) Pub. Date: Jun. 17, 2010

(54) Method of Cancer Treatment with Naphthol Analogs

(76) Inventors: Ja Seok Koo, Pearland, TX (US); William Bornmann, Missouri City, TX (US); David Maxwell, Pearland, TX (US)

Correspondence Address: WILLIAMS, MORGAN & AMERSON, 10333 RICHMOND, SUITE 1100, HOUSTON, TX, 77042, US

(21) Appl. No.: 12/529,252

(22) PCT Filed: Jan. 18, 2008
(86) PCT No.: PCT/US08/51398

§ 371 Date: Jan. 26, 2010

Related U.S. Application Data
(60) Provisional application No. 60/892,294, filed on Mar. 1, 2007.

(51) Int. Cl. A61K 31/66

(52) U.S. Cl. 514/119

(57) ABSTRACT

In one embodiment, the present invention relates to a method of treating a cancer characterized by overexpression of cyclic adenosine monophosphate response element-binding protein (CREB) or phosphorylated CREB (p-CREB) by administering to a patient suffering from the cancer a pharmaceutically-effective amount of a composition comprising a compound selected from the group consisting of 2-{1-[3-(Amidinothio)propyl]-1H-indol-3-yl}-3-(1-methylindol-3-yl)maleimide, naphthol analogs having the structure (I), wherein R¹ is selected from the group consisting of —H, —F, —Cl, —Br, and —I; R² is selected from the group consisting of —H, —F, —Cl, —Br, —I, —C₁₋₆ alkyl, —C₁₋₆alkenyl, and —C₁₋₆ alkynyl; R³ is selected from the group consisting of —H and —NO₂; and R⁴ is selected from the group consisting of —H, —C₁₋₆ alkyl, —C₁₋₆ alkenyl, —C₁₋₆ alkynyl, —OC₁₋₆ alkyl, —OC₁₋₆ alkenyl, and —OC₁₋₆ alkynyl; and salts and esters thereof.

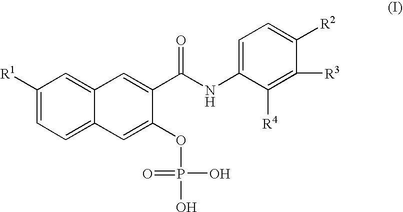

The United States government may own rights in the present invention pursuant to grant number 1R01HL077556 from the National Heart, Lung, and Blood Institute (NHLBI) of the National Institutes of Health.

### BACKGROUND OF THE INVENTION

The present invention relates generally to the field of cancer treatment. More particularly, it concerns chemotherapeutic treatments for cancers characterized by overexpression of cyclic adenosine monophosphate response element-binding protein (CREB) or phosphorylated CREB (p-CREB).

Lung cancer is the leading cause of cancer deaths, and its incidence is rising. In the United States, 174,470 new cases of lung cancer and 162,460 deaths from lung cancer were estimated for of this disease were expected in 2006. Non-small cell lung cancer (NSCLC) is an aggressive lung cancer that, despite standard treatment strategies such as surgery, radiotherapy, chemotherapy, or a combination of the three, is associated with a poor survival rate. Thus, there is a great need for better therapies for NSCLC. One tactic that looks promising to improve NSCLC treatment is modulating the expression of transcription factors associated with aggressive tumor growth and decreased patient survival. Research during the past few years has indicated that a transcription factor, the cyclic adenosine monophosphate (cAMP) response element-binding (CREB) protein regulates the expression of various genes such as Bcl-2, Bcl-X_{L}, cyclo-oxygenase-2, tumor necrosis factor alpha, that play an important role in cell survival, inflammation, and proliferation.

CREB has also been shown to be involved in the carcinogenesis of several types of cancer. For example, pharmacological inhibition of CREB activity has antiproliferative effects on cancer cells by inhibiting aromatase expression and estrogen production in breast adipose tissue. In pancreatic cells, CREB binds to specific DNA regulatory elements in the somatostatin gene associated with cellular processes such as islet cell differentiation and tumorigenesis. Further, the ectopic expression of dominant-repressor CREB (KCREB) demonstrated that the anti-apoptotic effects occurs through the phosphorylation of CREB in dihydrotestosterone stimulated, androgen-dependent prostate cancer cells, while CREB is constitutively active and has an anti-apoptotic effect in androgen-independent prostrate cancer cells. CREB is also overexpressed in bone marrow cells from patients with acute lymphoid or myeloid leukemia, suggesting that CREB is involved in leukemogenesis and that its expression can be a biomarker for leukemia. Finally, in human melanoma cells, CREB is a mediator of tumor growth and metastasis. Whereas the expression of KCREB, which targets the activating transcription factor-2 with its peptide fragment, sensitizes melanomas to apoptosis and inhibits tumor growth and metastasis in human melanoma cells.

Although it is not known whether CREB is directly involved in lung cancer, there are several lines of evidence that suggest that it is. For example, in an animal study demonstrated that the transgenic over expression of insulin-like growth factor-II in murine lung epithelium induced spontaneous lung tumors that displayed morphological characteristics similar to those of human pulmonary adenocarcinoma and that contained high levels of phosphorylated CREB. In addition, the increased expression of fibronectin in lung carcinoma was associated with the increased expression of CREB. Moreover, NSCLC cells often overexpress tumor necrosis factor-alpha, Bcl-X_{L}, Bcl-2, and interleukin-4, which are all regulated by CREB, and their suppression can result in apoptosis.

New approaches, such as developing new molecular targets for chemotherapy and chemoprevention, are urgently needed to improve control of various cancers, such as non-small cell lung cancer (NSCLC).

### SUMMARY OF THE INVENTION

In one embodiment, the present invention relates to a method of treating a cancer characterized by overexpression of cyclic adenosine monophosphate response element-binding protein (CREB) or phosphorylated CREB (p-CREB) by administering to a patient suffering from the cancer a pharmaceutically-effective amount of a composition comprising a compound selected from the group consisting of 2-{1-[3-(Amidinothio)propyl]-1H-indol-3-yl}-3-(1-methylindol-3-yl)maleimide, naphthol analogs having the structure I:

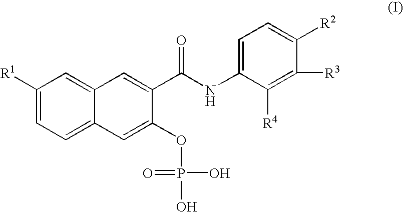

wherein R¹ is selected from the group consisting of —H, —F, —Cl, —Br, and —I;

R² is selected from the group consisting of —H, —F, —Cl, —Br, —I, —C₁₋₆ alkyl, C₁₋₆ alkenyl, and —C₁₋₆ alkynyl;

R³ is selected from the group consisting of —H and —NO₂; and

R⁴ is selected from the group consisting of —H, —C₁₋₆ alkyl, —C₁₋₆ alkenyl, —C₁₋₆ alkynyl, —OC₁₋₆ alkyl, —OC₁₋₆ alkenyl, and —OC₁₋₆ alkynyl;

and salts and esters thereof.

In another embodiment, the present invention relates to a composition comprising a compound selected from the group consisting of 2-{1-[3-(Amidinothio)propyl]-1H-indol-3-yl}-3-(1-methylindol-3-yl)maleimide, naphthol analogs having the structure I:

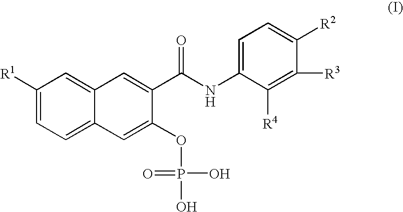

wherein R¹ is selected from the group consisting of —H, —F, —Cl, —Br, and —I; R² is selected from the group consisting of —H, —F, —Cl, —Br, —I, —C₁₋₆ alkyl, —C₁₋₆ alkenyl, and —C₁₋₆ alkynyl; R³ is selected from the group consisting of —H and —NO₂; and R⁴ is selected from the group consisting of —H, —C₁₋₆ alkyl, —C₁₋₆ alkenyl, —C₁₋₆ alkynyl, —OC₁₋₆ alkyl, —OC₁₋₆ alkenyl, and —OC₁₋₆ alkynyl; and salts and esters thereof; and a pharmaceutically-acceptable carrier.

The method provides a treatment for a number of cancers, including, but not limited to, non-small cell lung cancer (NSCLC), adenocarcinoma, head and neck cancer, breast cancer, liver cancer, colon cancer, brain cancer, leukemia, larynx cancer, tonsil cancer, thyroid gland cancer, kidney cancer, cervical cancer, uterine cancer, ovarian cancer, testicular cancer, prostate cancer, and gall bladder cancer. The composition is suitable for use in the method.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Expression of CREB and p-CREB in NHTBE and NSCLC cells. (A) Western blot analysis of CREB and p-CREB expression. Whole-cell lysates were isolated from fully differentiated NHTBE cells (lane 1) and 10 NSCLC cell lines (lane 2, H226; lane 3, H292; lane 4, H520; lane 5, H2170; lane 6, H1563; lane 7, H1734; lane 8, H1975; lane 9, H2228; lane 10, A549; lane 11, H1703) grown in optimal medium up to confluence. Equal amounts of lysate were subjected to Tris-Glycine sodium dodecyl sulfate-polyacrylamide gel (12%) electrophoresis and electrotransferred onto nitrocellulose membranes. Immunoblotting was performed with anti-CREB and anti-p-CREB antibodies. Equal protein loading was confirmed by stripping the blots and reprobing them with anti-β-actin antibody. (B) Quantification of the CREB and p-CREB signals shown in (A) presented as the ratio of the CREB or p-CREB signal in NSCLC cells to the CREB or p-CREB signal in NHTBE cells (which was set to 1). (C) qRT-PCR analysis of CREB mRNA expression. Total RNA from the NHTBE and NSCLC cells described in (A) was subjected to q-RT-PCR. The results are from a representative experiment performed twice, and samples were run in triplicate. The values are the ratios of the CREB mRNA expression in NSCLC cells to the CREB mRNA expression in NHTBE cells (which was set to 1), with mRNA levels normalized to the GAPDH mRNA expression level. The data are expressed as the mean±standard error (S.E.). *P<0.05, **P<0.01 v NHTBE cells (lane 1) using Student's t-test. (D) Q-PCR analysis of the CREB gene copy number. The genomic DNA sample of each cell was submitted to real-time PCR. The results are from a representative experiment performed twice, and samples were run in triplicate. The values are the ratios of the CREB DNA copy number in NSCLC cells to the CREB DNA copy number in NHTBE cells (which was set to 1) with DNA copy numbers normalized to the β-actin DNA level. The data are expressed as the mean±S.E. *P<0.05, **P<0.01 v NHTBE cells (lane 1) using Student's t-test.

FIG. 2. Expression of CREB and p-CREB in frozen human NSCLC tissue specimens and adjacent normal bronchial and bronchiolar epithelial specimens. (A) Western blot analysis of CREB and p-CREB expression. Soluble proteins were obtained from three squamous cell carcinoma (Sq) and three adenocarcinoma (Ad) tissue specimens (T) and paired matching normal tissue specimens (N) and to measure the levels of total CREB and p-CREB expression using Western blot analysis. Equal protein loading was confirmed by stripping the blots and reprobing them with an anti-β-actin antibody. (B) Quantification of the CREB and p-CREB signals shown in (A) presented as the ratio of the CREB or p-CREB signal in tumor tissue to the CREB or p-CREB signal from in normal tissue. CREB and p-CREB expression values in NHTBE cells (hatched bars) were used as controls and set to 1 to facilitate the calculation. (C) qRT-PCR analysis of CREB mRNA expression. Total RNA from the NSCLC tissue specimens and paired normal tissue specimens described in (A) was subjected to qRT-PCR. The results are from a representative experiment performed twice, and samples were run in triplicate. The values are the ratios of the CREB mRNA expression in normal and tumor tissue specimens to the CREB mRNA expression in NHTBE cells (which was set to 1), with CREB mRNA levels normalized to the GAPDH mRNA level. The data are expressed as the mean±S.E. *P<0.05, **P<0.01 v matched normal tissue using Student's t-test. (D) qPCR analysis of the CREB gene copy number. The genomic DNA extracted from the NSCLC tissue specimens and paired normal tissue specimens was submitted to qPCR. The results are from a representative experiment performed twice, and samples were run in triplicate. The values are the ratios of the CREB DNA copy number in normal and tumor tissue to the CREB DNA copy number in NHTBE cells (which was set to 1), with CREB DNA copy numbers normalized to the β-actin DNA level. The data are expressed as the mean±S.E. *P<0.05, **P<0.01 v matched normal tissue using Student's t-test.

FIG. 3. Immunohistochemical analysis of CREB and p-CREB expression in fixed adenocarcinoma and squamous cell carcinoma tissue specimens (center column) and adjacent normal bronchial and bronchiolar epithelial specimens (left column). Images were captured at a magnification of ×200. Arrows, nuclear CREB and p-CREB immunostaining in the basal layer of normal bronchial epithelial and tumor specimens. Representative images of CREB and p-CREB immunostaining of TMA specimens are shown in the right column (magnification, ×40).

FIG. 4. Kaplan-Meier curves of overall survival duration stratified according to CREB (top) and p-CREB (bottom) expression in the entire cohort (left panel) and in never-smokers (right panel). A total of 310 patients had information available for the survival analysis, and 94 deaths occurred. The median overall survival duration was 6.5 years, and the median follow-up duration was 3.2 years. The cut-off points for the CREB and p-CREB immunostaining scores were identified using martingale residual plots with respect to the overall survival duration. The curves are labeled with the corresponding immunostaining scores.

FIG. 5. BLiP plots of CREB and p-CREB immunostaining scores in fixed NSCLC tissue specimens (26 adenocarcinomas and 19 squamous cell carcinomas) and adjacent normal bronchial and bronchiolar epithelium.

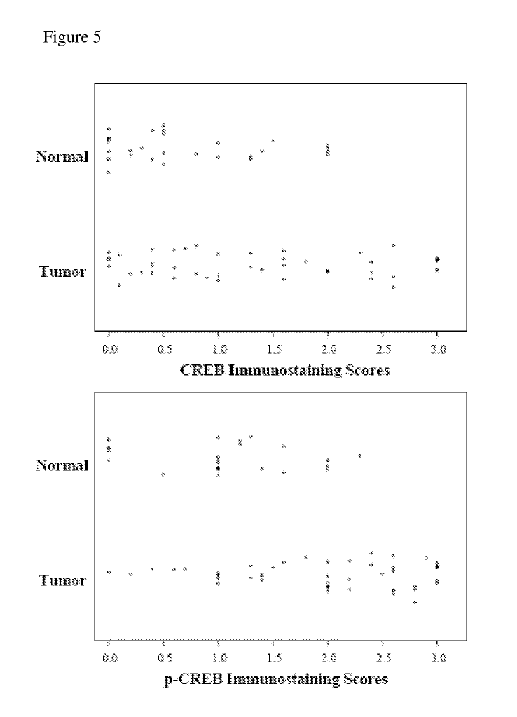

FIG. 6. Histograms of CREB and p-CREB immunostaining scores in TMA specimens by histologic subtype of NSCLC.

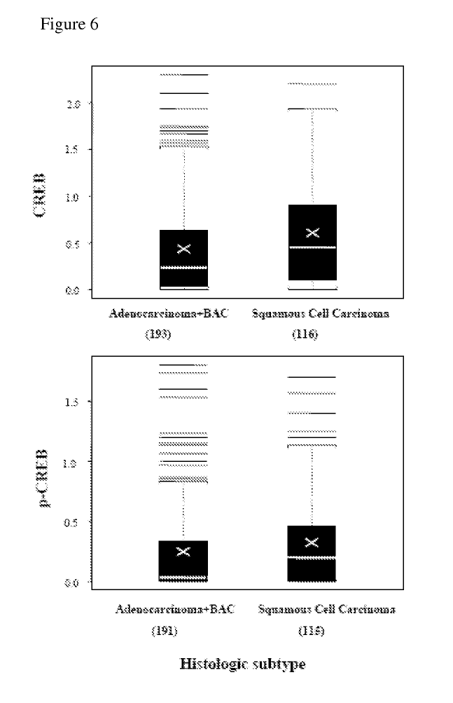

FIG. 7. NSCLC expresses constitutively active CREB. A, Whole cell lysates were made from NHTBE, H1734, H226, H292, A549 cells serum starved for 2 h and tested for constitutive levels of pCREB and total CREB by Western blot analysis. The same blot was stripped and re-probed with an anti-actin antibody to show total protein loading (lower panel). B, Nuclear extracts from NHTBE, H1734, A549, H226, and H292 cells serum starved for 2 h were prepared as described in Methods and then assayed for CREB activation by EMSA with a CRE consensus oligonucleotide-binding probe. C, for the supershift analysis, nuclear extracts were prepared from H1734 cells and incubated with pCREB, CREB, unlabeled (cold) CREB oligonucleotide probe, or mutant oligo, non specific Immunoglobin G (IgG) and then assayed for DNA binding to the CRE-site by EMSA. * Shifted band. The figures are three independent experiments.

FIG. 8. Constitutively active CREB is inhibited by Ro-31-8220 in NSCLC cells. A, Monolayer H1734 cells were treated with Ro-31-8220 in a serum free media for indicated times. Cells were also treated with various concentrations of Ro-31-8220 for 4 h in a serum free media. The whole extracts were subjected to Western blot analysis using the anti p-CREB. In each case, the same blot was stripped and re-probed with the anti-CREB antibody (lower panel in each figure). B, H1734 cells were treated with Ro-31-8220 in a serum free media for the indicated times. Cells were also treated with various concentrations of Ro-31-8220 for 4 h in a serum free media. Nuclear extracts were then tested for CRE-binding by EMSA. The figure is typical of three independent experiments. C, Ro-31-8220 induced the disappearance of nuclear pCREB in NSCLC cells. H1734 cells were incubated with or without 20 μM Ro-31-8220 for 4 h in a serum free media and then analyzed for pCREB by immunocytochemistry. The green stain indicates the localization of pCREB, and the blue stain indicates the localization of the nucleus.

FIG. 9. NSCLC cells express constitutive levels of phospho-Rsk and phospho-Erk1/2. Nuclear and cytoplasmic extracts were made from NHTBE, H1734, H226, H292, and A549 cells and tested for A, constitutive levels of phospho-Rsk in the nuclear extracts and D, phospho-Erk1/2 in the cytoplasmic extracts by Western blot analysis. B and E, H1734 cells were treated with Ro-31-8220 in a serum free media for the indicated times. C and F, Cells were also treated with various concentrations of Ro-31-8220 for 4 h in a serum free media. The nuclear and cytoplasmic extracts were subjected to Western blot analysis by using anti-phospho-Rsk (B and C), anti-phospho-Erk1/2 (E and F) respectively. In each case, the same blot was stripped and re-probed with total Rsk and Erk1/2 antibodies (lower panel in each figure). The figures are typical of three independent experiments.

FIG. 10. CREB regulated anti-apoptotic genes are suppressed by Ro-31-8220. Whole-cell lysates from NHTBE, H1734, H226, H292, and A549 cells were tested for A, constitutive expression of Bcl-X_{L} and D, Bcl-2 by western blot analysis. B and E, H1734 cells were treated with Ro-31-8220 in a serum free media for the indicated times. C and F, Cells were also treated with various concentrations of Ro-31-8220 for 4 h in a serum free media respectively. The whole cell extracts were subjected to Western blot analysis by using anti-Bcl-X_{L} (B and C), anti-Bcl-2 (E and F). In each case, the same blot was stripped and re-probed with anti-actin antibody to show equal protein loading (lower panel in each figure).

FIG. 11. PKC inhibitor (Ro-31-8220) inhibits cell proliferation of NSCLC cells. Cells (A, H1734 B, H226 C, A549 and D, H292) were plated in 96-well plates and incubated in triplicate with medium or the indicated dose of Ro-31-8220 for different days. Cell proliferation was assessed using MTT assays. The results are shown as the means (±standard deviations) of three independent experiments. E and F, Cells (NHTBE, H1734, H226) were plated in 96-well plates and incubated in triplicate with medium or with indicated concentration of Ro-31-8220 for different days and cell proliferation was assessed using MTT assays. The results are shown as the means (±standard deviations) of three triplicate three independent experiments.

FIG. 12. PKC inhibitor (Ro-31-8220) leads G2/M cell cycle arrest followed by apoptosis in NSCLC cells. A, H1734 cells were synchronized in a serum free media for 12 h before treatment and then H1734 cells were incubated in the absence or presence of 10 μM Ro-31-8220 for the indicated times in 5% of serum containing medium. Then, the cells were washed, fixed, stained with Propidium Iodide, and analyzed for DNA content by flow cytometry. B, H1734 cells were incubated in the absence or presence of 20 μM Ro-31-8220 for the indicated times in 5% of serum containing medium. Total cell lysates were subjected to Western blot analysis by using anti-PARP, anti-caspase-9 antibodies. C, Suppression of Ro-31-8220 induced caspase-3 cleavage by caspase-3 inhibitor. H1734 cells were pre-incubated with and without caspase inhibitor Ac-DEVD-CHO 5 μM for 2 h and then treated with 20 μM Ro-31-8220 for 24 h. Then, cell extracts were prepared and analyzed for caspase-3 cleavage by Western blot analysis using an anti-caspase-3 antibody. Dimethyl sulfonyloxide was used as a vehicle control. D, The caspase-3 inhibitor protects cells from Ro-31-8220 induced cytotoxicity. H1734 cells were incubated with different concentrations of caspase inhibitor Ac-DEVD-CHO as indicated for 2 h and then treated with 20 μM Ro-31-8220. After 24 h, cell viability was determined by the MTT. The results are shown as the means (±standard deviations) percentage viability from triplicate cultures of three independent experiments.

FIG. 13. Ro-31-8220 inhibits colony formation. Suppression of CREB inhibits cell proliferation and induces apoptosis. A, The effects of Ro-31-8220 on NSCLC colony formation in soft agar are shown. H1734 and H226 cells were treated with different concentrations of Ro-31-8220.Cell colonies were counted after 28-day incubation at 37° C. in 5% CO₂ under light microscopy. Colonies greater than 60 μm were scored. Result is mean (±standard deviations) of triplicate three independent experiments. B, H1734 cells were transfected with CREBwt, KCREB, and CREB133 plasmid, then assayed for cell proliferation by the MTT assays. The results are shown as the means (±standard deviations) of triplicate three independent experiments. C, H1734 cells were transfected with siCREB and non-specific control pool siRNA (NS-siRNA), then assayed for cell proliferation by the MTT assays. The results are shown as the means (±standard deviations) of triplicate three independent experiments. D, TUNEL analysis was performed after 48 h of transfection with the indicated plasmids and treatment of cells with Ro-31-8220 (5 μM) for 48 h. Nuclei appear blue (DAPI stain), TUNEL-positive nuclei stain green. Magnification 200×. The results are shown as the mean of three independent experiments. E, TUNEL analysis was performed after 48 h of transfection with the indicated plasmids. Nuclei appear blue (DAPI stain), TUNEL-positive nuclei stain green. Magnification 200×. The results are shown as the mean of three independent experiments. F, CREB knockdown induce PARP activation. H1734 cells were transiently transfected with siCREB, siRNA-NS, CREBwt, KCREB, and CREB133 plasmid as described in Example 2, Materials and Methods. Three days after transfection, equal amounts of whole-cell lysates were prepared and subjected to Western blot analysis using the indicated antibodies.

FIG. 14. NSCLC expresses constitutively active CREB. Supplemental to FIG. 7.

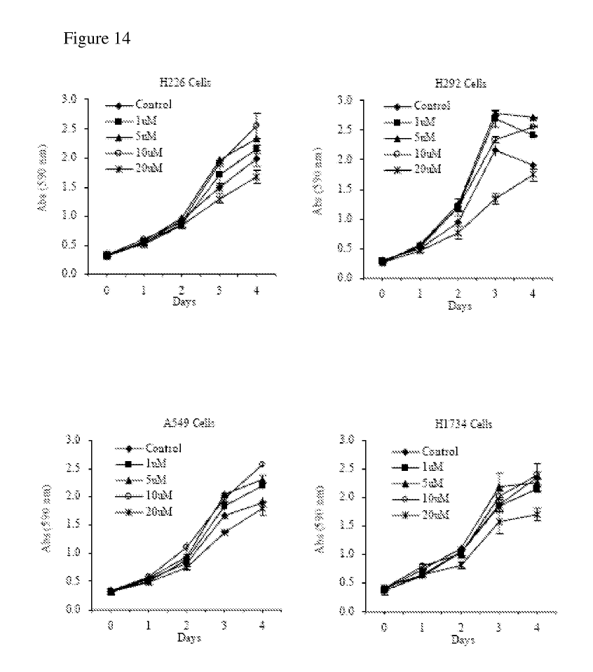

### DESCRIPTION OF ILLUSTRATIVE EMBODIMENTS

In one embodiment, the present invention relates to a method of treating a cancer characterized by overexpression of cyclic adenosine monophosphate response element-binding protein (CREB) or phosphorylated CREB (p-CREB) by administering to a patient suffering from the cancer a pharmaceutically-effective amount of a composition comprising a compound selected from the group consisting of 2-{1-[3-(Amidinothio)propyl]-1H-indol-3-yl}-3-(1-methylindol-3-yl)maleimide, naphthol analogs having the structure I:

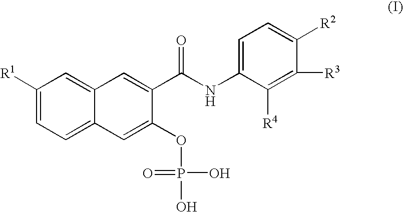

wherein R¹ is selected from the group consisting of —H, —F, —Cl, —Br, and —I;

R² is selected from the group consisting of —H, —F, —Cl, —Br, —I, —C₁₋₆ alkyl, —C₁₋₆ alkenyl, and —C₁₋₆ alkynyl;

R³ is selected from the group consisting of —H and —NO₂; and

R⁴ is selected from the group consisting of —H, —C₁₋₆ alkyl, —C₁₋₆ alkenyl, —C₁₋₆ alkynyl, —OC₁₋₆ alkyl, —OC₁₋₆ alkenyl, and —OC₁₋₆ alkynyl;

and salts and esters thereof.

“Treatment” is used herein, when referring to cancer, to any procedure fatal, either directly or indirectly, to at least one cancer cell characterized by overexpression of CREB or p-CREB in vivo. A number of cancers characterized by overexpression of CREB or p-CREB are known at this time, although the present invention is not limited to such known cancers. In one embodiment, the cancer is selected from the group consisting of non-small cell lung cancer (NSCLC), adenocarcinoma, head and neck cancer, breast cancer, liver cancer, colon cancer, brain cancer, leukemia, larynx cancer, tonsil cancer, thyroid gland cancer, kidney cancer, cervical cancer, uterine cancer, ovarian cancer, testicular cancer, prostate cancer, and gall bladder cancer. In one particular embodiment, the cancer is NSCLC.

In one embodiment, the compound is a naphthol analog having structure I. Any naphthol analog having structure I can be used in the method. In one embodiment, the naphthol analog is selected from the group consisting of the compound having structure I wherein R¹ is —H, R² is —Cl, R³ is —H, and R⁴ is —H (naphthol AS-E phosphate); the compound having structure I wherein R¹ is —Br, R² is —H, R³ is —H, and R⁴ is —OCH₃ (naphthol AS-BI phosphate); the compound having structure I wherein R¹ is —H, R² is —H, R³ is —NO₂, and R⁴ is —H (naphthol AS-BS phosphate); the compound having structure I wherein R¹ is —H, R² is —CH₃, R³ is —H, and R⁴ is —CH₃ (naphthol AS-MX phosphate); and the compound having structure I wherein R¹ is —H, R² is —Cl, R³ is —H, and R⁴ is —CH₃ (naphthol AS-TR phosphate). In a further embodiment, the naphthol analog is naphthol AS-E phosphate.

Generally, the naphthol analog is unlikely to be the sole ingredient of the composition, though it may be. In one embodiment, the composition further comprises a pharmaceutically-acceptable carrier. By “pharmaceutically-acceptable” is meant that the carrier is suitable for use in medicaments intended for administration to a patient. Parameters which may considered to determine the pharmaceutical acceptability of a carrier can include, but are not limited to, the toxicity of the carrier, the interaction between the naphthol analog and the carrier, the approval by a regulatory body of the carrier for use in medicaments, or two or more of the foregoing, among others.

The compositions can be made up in any conventional form known in the art of pharmaceutical compounding. Exemplary forms include, but are not limited to, a solid form for oral administration such as tablets, capsules, pills, powders, granules, and the like. In one embodiment, for oral dosage, the composition is in the form of a tablet or a capsule of hard or soft gelatin, methylcellulose, or another suitable material easily dissolved in the digestive tract.

Typical preparations for intravenous administration would be sterile aqueous solutions including water/buffered solutions. Intravenous vehicles include fluid, nutrient and electrolyte replenishers. Preservatives and other additives may also be present.

In the administering step, the composition can be introduced into the patient by any appropriate technique. An appropriate technique can vary based on the patient, the location and stage of the cancer, and the components of the composition, among other parameters apparent to the skilled artisan having the benefit of the present disclosure. Administration can be systemic, that is, the composition is not directly delivered to a tissue, tissue type, or organ at which the cancer is present, or it can be localized, that is, the composition is directly delivered to a tissue, tissue type, or organ at which the cancer is present. The route of administration can be varied, depending on the composition and the cancer, among other parameters, as a matter of routine experimentation by the skilled artisan having the benefit of the present disclosure. Exemplary routes of administration include transdermal, subcutaneous, intravenous, intraarterial, intramuscular, intrathecal, intraperitoneal, oral, rectal, and nasal, among others. In one embodiment, the route of administration is oral or intravenous.

A pharmaceutically-effective amount of the composition is one that imparts a dosage sufficient to treat the cancer. In one embodiment, administering comprises a dosage from about 1 μg naphthol analog/kg body weight per day to about 1 g naphthol analog/kg body weight per day.

A regimen for treating cancer will typically involve multiple dosages. In one embodiment, the administering step is repeated once every two to three days for a period of from about three to about twelve weeks. Other treatment regimens are possible and can be routinely selected by the ordinary skilled artisan.

In another embodiment, the present invention relates to a composition comprising a compound selected from the group consisting of 2-{1-[3-(Amidinothio)propyl]-1H-indol-3-yl}-3-(1-methylindol-3-yl)maleimide, naphthol analogs having the structure I:

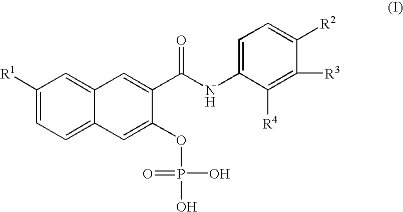

wherein R¹ is selected from the group consisting of —H, —F, —Cl, —Br, and —I; R² is selected from the group consisting of —H, —F, —Cl, —Br, —I, —C₁₋₆ alkyl, —C₁₋₆ alkenyl, and —C₁₋₆ alkynyl; R³ is selected from the group consisting of —H and —NO₂; and R⁴ is selected from the group consisting of —H, —C₁₋₆ alkyl, —C₁₋₆ alkenyl, —C₁₋₆ alkynyl, —OC₁₋₆ alkyl, —OC₁₋₆ alkenyl, and —OC₁₋₆ alkynyl; and salts and esters thereof; and a pharmaceutically-acceptable carrier.

The naphthol analog and the pharmaceutically-acceptable carrier can be as described above.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### The Influence of CREB and p-CREB Expression on the Survival of Non-Small Cell Lung Cancer Patients

Purpose

We conducted this study to evaluate the transcription factor cyclic adenosine monophosphate response element-binding protein (CREB) for its role in the pathogenesis of non small cell lung cancer (NSCLC) and its prognostic importance of CREB in NSCLC patients.

Methods

We used western blotting to evaluate the expressions of CREB and phosphorylated CREB (p-CREB) were evaluated in normal human tracheobronchial epithelial cells (NHTBE), NSCLC cell lines, and banked NSCLC tissue specimens. Immunohistochemistry of tissue microarray slides was performed. The relationships of CREB or p-CREB immunostaining scores to the survival rate by smoking status were assessed in patients with NSCLC using Kaplan-Meier survival curves and Cox proportional hazards models.

Results

CREB and p-CREB were significantly overexpressed in most NSCLC cell lines and tissue specimens compared with expressions in NHTBE cells and paired adjacent normal tissue specimens. Analyses of CREB mRNA and the CREB gene copy number in cultured cells and tissues showed that CREB overexpression occurred at the transcriptional level. Overexpressions of CREB and p-CREB and survival were inversely related in NSCLC patients, although this relationship was driven by smoking status. A significantly worse survival rate was associated with overexpression of CREB or p-CREB only in never smokers (not in current or former smokers).

Conclusion

CREB and p-CREB are overexpressed mainly at the transcriptional level in NSCLC cells and tissues. These overexpressions correlated with a poor survival rate in patients, especially never smokers, with NSCLC. These results are the first of their kind in NSCLC and illustrate the potential of CREB as a new molecular target for the prevention and treatment of NSCLC.

Introduction

Lung cancer is the leading cause of cancer deaths, and its incidence is rising.¹ In the United States, 174,470 new cases of lung cancer and 162,460 deaths from lung cancer were estimated for of this disease were expected in 2006.² New approaches, such as developing new molecular targets for NSCLC chemotherapy and chemoprevention, are urgently needed to improve control of deadly lung cancer.

Cyclic adenosine monophosphate response element-binding protein (CREB) has been shown to play important roles in cell differentiation,³ survival,^{4,5} proliferation,⁶ and development.^{7,8} CREB is activated by multiple signaling pathways, including the cAMP/protein kinase A, phosphatidylinositol 3-kinase/Akt, extracellular signal-regulated kinase/p90 ribosomal S6 kinase, and p38/mitogen- and stress-activated protein kinase pathways, stimulation by cAMP, growth factors, hormones, and cytokines.⁹ Once activated, CREB induces the expression of cAMP response element-containing target genes,¹⁰⁻¹² involved in the cell cycle,¹³ apoptosis suppression,¹⁴ proliferation,¹⁵ neovascularization,¹⁶ inflammation,¹⁷ and tumorigenesis.¹⁸

CREB overexpression was shown in certain tumor types. CREB expression was higher in bone marrow cells of patients obtained from patients with acute lymphoid and myeloid leukemia than in cells of leukemia patients either in remission or with no evidence of disease.¹⁹⁻²¹ CREB transcripts were expressed more highly in breast tumors than in normal breast adipose tissue.²² Among studies examining the role of CREB in lung cancer to date, only recent studies have shown elevated expression of phosphorylated CREB (p-CREB) in lung tumors generated in MMTV-IGF II transgenic mice. Elevated expression of phosphorylated CREB (p-CREB) has only recently been shown in lung tumors, which were generated in MMTV-IGF-II transgenic mice ^{23,24} and that dominant-negative CREB (KCREB) also recently was shown to significantly inhibit the growth of adenocarcinoma cell lines in vitro.^{25,26} Whether CREB expression is altered in human NSCLC tumors or CREB/p-CREB expression correlates inversely with survival in NSCLC patients, however, has not been previously shown.

Based on these previous CREB findings in the lung and other cancer sites, we investigated whether CREB is a regulatory factor in NSCLC development. For this purpose, we measured the expression and activation status of CREB and analyzed the CREB gene copy number in 10 NSCLC cell lines and 6 frozen NSCLC tissue specimens and compared them with these CREB features in normal human tracheobronchial epithelial (NHTBE) cells and paired normal tissue specimens, respectively. We also analyzed CREB and p-CREB expression in 45 paraffin-embedded whole specimens of NSCLC tumor and adjacent normal bronchial/bronchiolar epithelial tissue. Last, we assessed the levels of CREB and p-CREB in association with clinicopathological parameters and overall survival of 310 NSCLC patients with banked tissue available for tissue microarray (TMA) analysis.

Patients and Methods

NSCLC Tissue Specimens and Construction of Tumor TMAs

Archival formalin-fixed, paraffin-embedded specimens of NSCLC tumor and adjacent normal lung tissue surgically resected primary lung tumors were obtained from the tissue bank of the M. D. Anderson Cancer Center Lung Cancer SPORE. These specimens originally were collected from NSCLC patients who had undergone lobectomies and pneumonectomies of primary lung tumors. All tumors were histologically examined and classified using the 2004 World Health Organization classification.²⁷ Of a total of 335 NSCLCs resected in our institution from 1999 to 2001, 263 NSCLCs were available and used for TMA construction. In addition, 79 NSCLC cases were included in the TMA analysis to enrich our study population with patients with lung cancer who were never-smokers or long-term former smokers. They consisted of 11 and 68 cases surgically resected in our institution from 1994 to 1996 and from 2002 to 2004, respectively. Thus, a total of 342 NSCLCs (223 adenocarcinomas and 119 squamous cell carcinomas) were included in the TMA. In the TMAs, CREB and p-CREB immunohistochemistry was evaluable in 310 NSCLC specimens (194 adenocarcinomas and bronchioloalveolar carcinomas (BAC) and 116 squamous cell carcinomas). After histologic examination of NSCLC tumor specimens, tumor TMA was prepared using triplicate 1-mm-diameter cores per tumor to obtain tissue from central, intermediate, and peripheral tumor areas.

Forty-five surgically resected NSCLCs (26 adenocarcinomas and 19 squamous cell carcinomas) containing adjacent normal bronchial/bronchiolar epithelia were randomly selected for immunohistochemistry of CREB and p-CREB. The M. D. Anderson Cancer Center Institutional Review Board approved the use of the archival clinical tissue specimens. Patients who had smoked at least 100 cigarettes in their lifetime were defined as smokers, and smokers who quit smoking at least 12 months before their lung cancer diagnosis were defined as former-smokers.

Cell Cultures

NHTBE cells from Cambrex (Walkersville, Md.) were organotypically cultured and maintained as described previously. ^{11,28-33} Ten NSCLC cell lines were obtained from the American Type Culture Collection (Manassas, Va.) and maintained in RPMI 1640 medium containing 10% fetal bovine serum and gentamicin (0.1%).

Western Blot Analysis

Whole cell extracts from the NHTBE cells, the NSCLC cell lines, and archival frozen six NSCLC specimens with paired normal tissue specimens were subjected to Western blotting to measure the expression of CREB and p-CREB as described previously.³⁰

CREB mRNA and Gene Copy Number

Total RNA and genomic DNA from the NHTBE cells, NSCLC cells, and frozen NSCLC specimens were extracted using the QIAGEN RNeasy Mini Kit and Blood & Cell Culture DNA Mini Kit (QIAGEN, Valencia, Calif.) and subjected to quantitative real-time polymerase chain reaction (qRT-PCR) and quantitative polymerase chain reaction (qPCR) analysis as described previously.³⁴ The primer sequences used for detection of CREB mRNA were: forward, 5′-ACTGTAACGGTGCCAACTCC-3′; reverse, 5′-GAATGGTAGTACCCGGCTGA-3′. For endogenous control of qRT-PCR, human glyceraldehyde-3-phosphate dehydrogenase (GAPDH) labeled with VIC dye (Applied Biosystems, Foster City, Calif.) was used. Primer sequences used for detection of the CREB gene copy number were: forward, 5′-AAGAGGAGACTTCAGCACCTG-3′; reverse, 5′-GCAAAACTGAGAAGACTTGGC-3′. For endogenous control of qPCR, β-actin primer sequences were used: forward, 5′-AGGTCATCACCATTGGCAAT-3′; reverse, 5′-AATGAGGGCAGGACTTAGCTT-3′.

Immunohistochemical Analysis

Immunohistochemical analysis of NSCLC specimens was performed using an anti-CREB or anti-p-CREB antibody (Upstate, Lake Placid, N.Y.) according to the manufacturer's instructions. Distinct nuclear immunostaining for CREB and p-CREB was quantified by an experienced lung cancer pathologist (I.I.W.) under a light microscope. In each specimen, up to 1000 tumor and epithelial cells were examined. The intensity of the CREB and p-CREB immunostaining was graded on a scale of 0 to 3:0, no staining; 1, weak; 2, moderate; and 3, strong. The extent of positive immunoreactivity for CREB or p-CREB (0 to 1; 1=100%) was calculated as the percentage of cells that had nuclear staining for CREB or p-CREB. Scoring of the staining was determined as the product of positive immunostaining intensity (0 to 3) and positive immunoreactivity extension (0 to 1).^{19,35}

Statistical Analysis

A mixed-effect general linear model was used to assess the differences in CREB and p-CREB expression between normal tissue and tumor tissue. The Kruskal-Wallis test and Wilcoxon rank sum test were used to assess the relationships between CREB and p-CREB expression in TMA specimens and patients' demographic and clinicopathological characteristics. Cox proportional hazards regression models were used to assess the effect of the CREB and p-CREB immunostaining scores on overall survival duration (time from surgery to death of any cause). Survival curves were estimated using the Kaplan-Meier product limit estimates, and differences between groups were assessed statistically using the log-rank test. The need for transformation of predictive variables in the Cox proportional hazards model was assessed using martingale residual plots. Predictive variables with P values less than 10 for the univariate Cox proportional hazards model were included in a multivariate model. In this multivariate model, backward elimination with a P value cut-off of 0.05 was used; any previously deleted variable was then allowed to re-enter the final model if P<0.05.

Results

CREB and p-CREB Overexpressed in NSCLC Cell Lines versus in NHTBE Cells

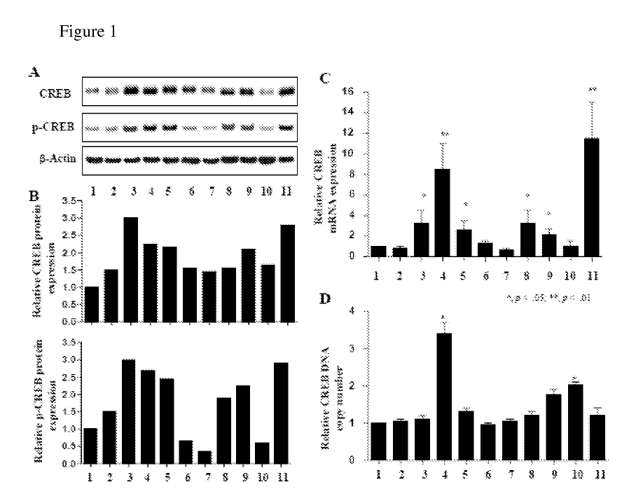

Western blot analysis showed that most NSCLC cell lines expressed higher levels of CREB (9 of 10 cell lines) and p-CREB (7 of 10 for p-CREB) protein than did NHTBE cells (FIGS. 1A and 1B). qRT-PCR analysis showed a similar pattern of CREB mRNA expression—about 2- to 12-fold higher in 6 of 10 NSCLC cell lines (versus in NHTBE cells) (P<0.01 in all 6) (FIG. 1C); these 6 cell lines also had the highest p-CREB protein expression levels.

Unlike CREB protein and mRNA, CREB gene copy number (qPCR analysis) was significantly increased in only 2 of the 10 NSCLC cell lines (P<0.05 for both cell lines) (FIG. 1D). Our data clearly show that CREB was overexpressed and highly activated in most of the NSCLC cell lines tested. Moreover, these overexpressions of CREB and p-CREB mainly resulted from increased CREB gene transcription, not from an amplified CREB gene copy number (versus in NHTBE cells).

CREB Overexpressed in Frozen Human NSCLC Tissue Versus in Adjacent Normal Tissue

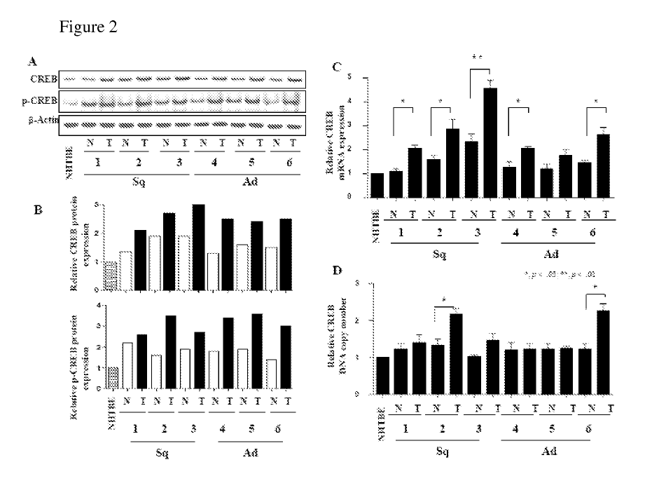

CREB protein was overexpressed and more activated in frozen tumor tissue versus in adjacent normal tissue (Western blot analysis) (FIGS. 2A and 2B). Of the 6 frozen specimens in our analysis (3 squamous cell carcinoma, 3 adenocarcinoma), CREB mRNA expression was significantly higher in all 3 squamous cell specimens and in 2 of the 3 adenocarcinoma specimens (versus in the adjacent normal tissue) (qRT-PCR) (P<0.05 for all 5 specimens) (FIG. 2C). qPCR analysis revealed that only one squamous cell carcinoma tissue specimen and one adenocarcinoma tissue specimen had a significantly increased DNA copy number when compared with normal tissue specimens (P<0.05 for both specimens) (FIG. 2D). The overexpression of CREB and p-CREB observed in these frozen NSCLC tissue specimens was concordant with our results in the NSCLC cell lines. Taken together, our results in the cell lines and frozen tissue specimens showed that the CREB overexpression in them occurred primarily at the gene transcription level but in some cases could have been a result of an increased CREB gene copy number.

CREB Overexpressed in Banked Paraffin-Embedded Human NSCLC Specimens Versus in Adjacent Normal Tissue Specimens

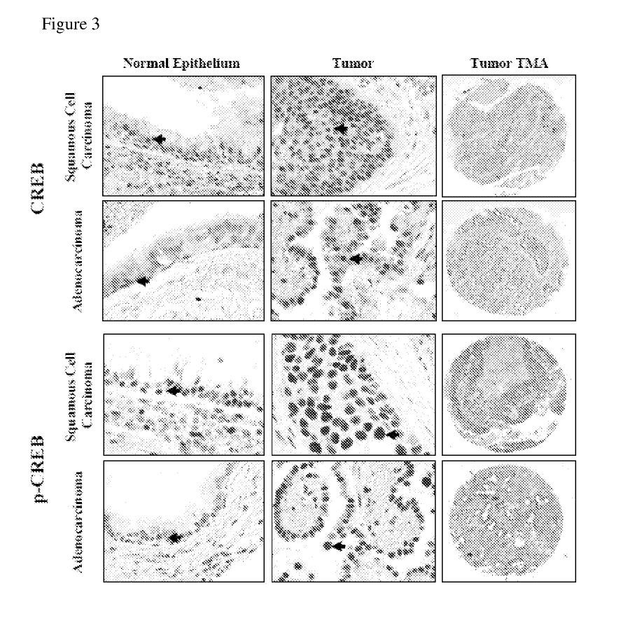

Immunohistochemical analysis of CREB and p-CREB expression in whole tissue sections of paraffin-embedded NSCLC specimens and adjacent normal bronchial and bronchiolar epithelium showed stronger nuclear staining for CREB and p-CREB in both adenocarcinoma and squamous cell carcinoma tissue than in normal epithelium (FIG. 3, left and middle columns). Statistical analysis showed significantly higher immunostaining scores in tumor tissue than in normal tissue for both CREB (1.37 v 0.73; P=0.013) and p-CREB (1.96 v 1.05; P=0.0002). The distributions of the staining scores for the tumor tissue and normal epithelium are shown in Supplemental FIG. 1.

Associations of CREB and p-CREB Expression with Histologic NSCLC Subtypes and NSCLC Patients' Demographic and Clinicopathological Characteristics

We performed immunohistochemical analyses of CREB and p-CREB expression in NSCLC TMAs containing 194 adenocarcinoma and BAC specimens and 116 squamous cell carcinoma specimens to assess potential associations of CREB and p-CREB overexpression with the demographic and clinicopathological characteristics of NSCLC patients and histologic subtypes of NSCLC listed in Table 1. Representative images of the staining of CREB and p-CREB in the TMA specimens are shown in FIG. 3 (right-hand column).

According to the Wilcoxon rank sum test, the CREB and p-CREB immunostaining scores were significantly higher in the squamous cell carcinoma specimens than in the adenocarcinoma and BAC specimens (CREB: 0.61 v 0.43, P=0.002; p-CREB: 0.32 v 0.25, P=0.008) (Table 2 and Supplemental FIG. 2). Levels of immunohistochemical expression of CREB and p-CREB were lower in TMA than in whole specimens. This phenomenon probably was due to larger tissue areas and stronger immunostaining in the whole sections than in the TMAs. We detected no significant differences in CREB or p-CREB expression between other demographic and clinicopathological subpopulations (Supplemental Table 1).

Effects of CREB and p-CREB Overexpression on Survival Overall and by Smoking Status

Overall Survival

Overexpression of CREB (immunostaining score >0.9) or p-CREB (immunostaining score >0.7) was significantly associated with shorter overall survival of NSCLC patients (log-rank test, P=0.02 and P=0.002, respectively). We based cut-off points for the immunostaining score on martingale residual plots.

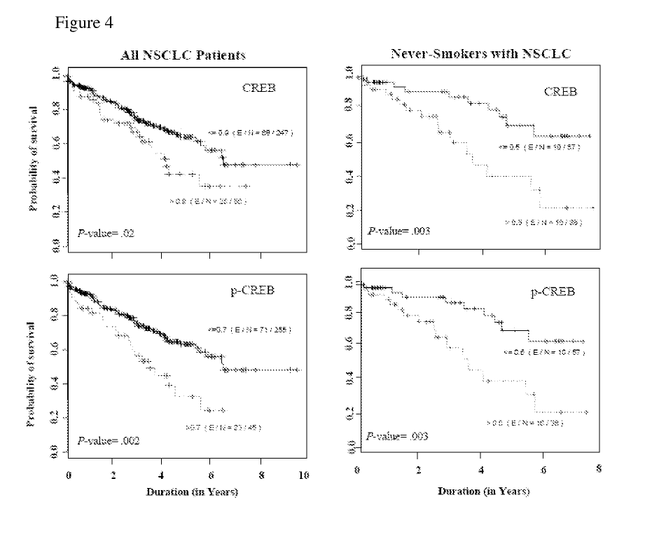

The Kaplan-Meier survival curves shown in FIG. 4 demonstrate that overexpression of CREB or p-CREB was related to a low probability of survival. We also used univariate Cox proportional hazards models to estimate the effects of covariates on overall survival duration. Factors that significantly affected overall survival were age, histologic subtype, p-CREB expression, pathological T classification, pathological N classification, and pathological TNM stage (Supplemental Table 1). Multivariate Cox proportional hazards regression model analysis showed that the expression of CREB and p-CREB was significantly associated with overall survival after accounting for the effects of age and pathological T and N classification (P=0.01). One-unit increases in the CREB and p-CREB immunostaining scores increased the risk of death by 51% and 80%, respectively (Supplemental Table 2).

Survival by Smoking Status

The inverse relationship between the level of CREB or p-CREB expression and the probability of survival was intensely dependent on smoking status. Overexpression of CREB and p-CREB significantly lowered the probability of survival in never-smokers (FIG. 4, right-hand panels) but not in former- or current-smokers (data not shown). Also, univariate Cox proportional hazards model analysis demonstrated that both CREB and p-CREB immunostaining scores significantly affected overall survival in never-smokers, with increasing the risk of death by 73% (CREB) and 169% (p-CREB), respectively (Table 3).

Discussion

The present and first comprehensive study of the role of CREB in the development and pathogenesis of NSCLC showed that CREB is overexpressed and highly constitutively activated in NSCLC cell lines versus in NHTBE cells. CREB and p-CREB also were overexpressed in frozen NSCLC tumor specimens versus in paired normal tissue specimens. This overexpression of CREB protein resulted primarily from the transcriptional overexpression of CREB mRNA rather than from amplification of the CREB gene.

These results are consistent with a recent report that CREB expression is upregulated at both the protein and mRNA level in primary acute myeloid leukemia cells (versus in normal cells).²¹ This report, however, indicates that CREB overexpression was associated mainly with increased CREB gene copy number (in 3 of 4 acute myeloid leukemia patients), whereas we found that CREB and p-CREB overexpression occurred mainly at the gene transcription level (and possibly as a result of an increased DNA copy number in a few cases).

Immunohistochemical analysis of slides containing sections of whole tissue specimens confirmed that expressions of CREB and p-CREB were significantly higher in NSCLC specimens than in adjacent normal bronchial and bronchiolar epithelium. Our immunohistochemical study results in NSCLC TMAs showed that CREB and p-CREB immunostaining scores were significantly higher in the squamous cell carcinoma specimens than in the adenocarcinoma including BAC specimens.

Kaplan-Meier survival curves revealed that CREB and p-CREB overexpressions were significantly associated with worse survival in the entire cohort of patients with NSCLC. This overall effect on survival, however, was driven by CREB and p-CREB overexpression in never smokers. The survival duration in never-smokers was strongly influenced by the levels of CREB and p-CREB, whereas the duration in former- and current-smokers were less or not affected by them. These data suggest that the levels of CREB and p-CREB could be a useful biomarker for prediction of survival duration for never-smokers with NSCLC but not for ever-smokers with this disease.

The biology of lung cancer differs in never-smokers and ever-smokers,^{36,37} and this difference seems to favor never-smokers in terms of prognosis and treatment outcome of NSCLC.^{37,38,39-41} It is unclear, however, why overexpression of CREB and p-CREB is a negative prognostic factor in never-smokers but not in ever-smokers is currently unclear. One possible explanation is that survival of cancer cells may depend substantially on CREB activity substantially in never-smokers but less so in former-smokers and not at all in current-smokers. Tobacco smoke is a well-known procarcinogen that induces numerous genetic and molecular changes. Former- and current-smokers may have other various compounding factors that predominate over CREB activity in affecting overall survival.

However, abnormal overexpression of CREB and p-CREB may also cause numerous changes in the cells of ever-smokers. Thus, further studies are required to determine the role of CREB in lung carcinogenesis in former and current smokers. In fact, a study recently showed that transcriptional activity of CREB mediates tobacco smoke-stimulated overexpression of amphiregulin,⁴² which is a ligand of the epidermal growth factor receptor, a tumor promoter for oral squamous cell carcinoma.⁴³ Growth factors like amphiregulin affect the growth and survival of cancer cells. Researchers also showed that overexpression of amphiregulin was associated with poor prognosis in patients with NSCLC.⁴⁴ These provocative data highlight the need for further studies to determine the role of CREB in lung carcinogenesis in former and current smokers.

CREB's ability to stimulate the expression of CREB target genes involved in cell growth, cell cycle progression, and suppression of apoptosis may play a role in the development of lung cancer^{13,14} and metastasis of tumors.^{18,45,46}

CREB overexpression may lead to upregulation of genes or activation of pathways that support tumor growth and survival. In support of this hypothesis, we have unpublished data indicating that inactivation or reduction of CREB (via a dominant repressor of CREB or small interfering RNA targeting CREB) reduced the expression of antiapoptotic genes and consequently inhibited the growth and survival of NSCLC cells. The finding in our present study that increased expression of CREB and p-CREB correlated with decreased overall survival durations suggests that sustained hyperactive CREB in malignant cells supports the growth and survival of tumor cells.

The present study provides important new insights into the role of the transcription factor CREB in the development and pathogenesis of NSCLC: CREB was overexpressed and highly constitutively active in NSCLC cell lines and banked NSCLC tumor specimens: CREB overexpression appears to be related more to increased RNA transcription than gene amplification suggesting that CREB expression and/or activation is a correctable therapeutic target in NSCLC; and overexpressions of CREB and p-CREB were independently correlated with significantly shortened overall survival durations in never-smokers but not ever-smokers with NSCLC.

To the best of our knowledge, our study provides evidence for the first time that overexpression of CREB and p-CREB and smoking status are negative prognostic factors in NSCLC patients. Therefore, targeting CREB, such as with CREB inhibitors may be a preventive and/or therapeutic strategy for the management of patients, especially never-smokers with NSCLC.

TABLE 1
Demographic and Clinicopathological Characteristics of Patients
with NSCLC Whose Tissue Specimens Were Examined for CREB
and p-CREB Expression Using TMA Immunohistochemical
Analysis (n = 310)

| Characteristic | No. (%) | |
|---|---|---|
| Median age | 67.7 years | |
| Sex | | |
| Female | 164 | (53.6) |
| Male | 142 | (46.4) |
| Race | | |
| White | 276 | (89.0) |
| Other | 34 | (11.0) |
| Smoking status | | |
| Never | 96 | (32.7) |
| Former | 122 | (41.5) |
| Current | 76 | (25.9) |
| Time between quitting smoking and surgery (n = 198)* | | |
| 0-14 days | 22 | (11.1) |
| >14 days to ≦1 month | 24 | (12.1) |
| >1 month to ≦12 months | 30 | (15.2) |
| >12 months to ≦5 years | 101 | (51.0) |
| >5 years | 21 | (10.6) |
| Histologic subtype | | |
| Squamous cell carcinoma | 116 | (37.4) |
| Adenocarcinoma and BAC | 194 | (62.6) |
| Pathological T classification (n = 278) | | |
| T1 | 102 | (36.7) |
| T2 | 154 | (55.4) |
| T3 | 14 | (5.0) |
| T4 | 8 | (2.9) |
| Pathological N classification (n = 277) | | |
| N0 | 199 | (71.8) |
| N1 | 50 | (18.1) |
| N2 | 28 | (10.1) |
| N3 | 0 | (0.0) |
| Pathological M classification (n = 279) | | |
| M0 | 274 | (98.2) |
| M1 | 5 | (1.8) |
| Pathological TNM stage** (n = 279) | | |
| I | 180 | (64.5) |
| II | 59 | (21.1) |
| III | 35 | (12.5) |
| IV | 5 | (1.8) |

*Former and current smokers only.
**Pathological TNM stage I to IV were classified according to the Revised International System for Staging Lung Cancer.⁴⁷

TABLE 2
CREB and p-CREB Immunostaining Scores in NSCLC TMA Specimens

| | | Immunostaining Score | | | | |
|---|---|---|---|---|---|---|
| Tissue Type | No. | Mean ± S.D. | Median | Minimum | Maximum | P Value* |
| Adenocarcinoma | | | | | | |
| and BAC | | | | | | |
| CREB | 193 | 0.43 ± 0.52 | 0.23 | 0 | 2.3 | .002† |
| p-CREB | 191 | 0.25 ± 0.40 | 0.03 | 0 | 1.8 | .008† |
| Squamous cell | | | | | | |
| carcinoma | | | | | | |
| CREB | 116 | 0.61 ± 0.54 | 0.45 | 0 | 2.2 | — |
| p-CREB | 115 | 0.32 ± 0.40 | 0.20 | 0 | 1.7 | — |

Abbreviation:
S.D., standard deviation.
*P value generated from a mixed-effect model.
†P value from a Wilcoxon rank sum test comparing CREB and p-CREB per histologic subtype adenocarcinoma plus BAC ν squamous cell carcinoma.

TABLE 3
Estimation of Overall Survival Durations in Patients with NSCLC
per Smoking Status Using Univariate Cox Proportional Hazards
Regression Models

| | Parameter | Standard | | Hazard |
|---|---|---|---|---|
| Smoking Status | Estimate | Error | P Value | Ratio |
| Never-Smokers | | | | |
| CREB | 0.55 | 0.24 | 0.02 | 1.73 |
| p-CREB | 0.99 | 0.43 | 0.02 | 2.69 |
| Former Smokers | | | | |
| CREB | 0.24 | 0.28 | 0.39 | 1.28 |
| p-CREB | 0.75 | 0.30 | 0.01 | 2.13 |
| Current Smokers | | | | |
| CREB | −0.46 | 0.56 | 0.41 | 0.63 |
| p-CREB | −0.03 | 0.60 | 0.96 | 0.97 |

SUPPLEMENTAL TABLE 1
Estimation of Overall Survival Durations in Patients with NSCLC
per Demographic and Clinicopathological Characteristics Using
Univariate Cox Proportional Hazards Regression Models

| | Parameter | | Hazard |
|---|---|---|---|
| Characteristic | Estimate ± S.E. | P Value | Ratio |
| Age | 0.04 ± 0.01 | .0002 | 1.04 |
| Sex (male ν female) | 0.34 ± 0.21 | .1000 | 1.40 |
| Race (white ν other) | −0.31 ± 0.34 | .3500 | 0.73 |
| Smoking status | | | |
| Former ν never | 0.24 ± 0.25 | .3300 | 1.27 |
| Current ν never | 0.03 ± 0.29 | .9300 | 1.03 |
| Histologic subtype (squamous cell | 0.65 ± 0.21 | .0020 | 1.92 |
| carcinoma ν adenocarcinoma/BAC) | | | |
| CREB expression | 0.28 ± 0.17 | .1000 | 1.33 |
| p-CREB expression | 0.66 ± 0.23 | .0040 | 1.93 |
| Pathological T classification | 0.97 ± 0.27 | .0002 | 2.63 |
| (T2 + T3 + T4 ν T1) | | | |
| Pathological N classification | 0.73 ± 0.23 | .0010 | 2.08 |
| (N1 + N2 + N3 ν N0) | | | |
| Pathological M classification | 1.05 ± 0.72 | .1400 | 2.87 |
| (M1 ν M0) | | | |
| Pathological TNM stage | | | |
| II ν I | 0.65 ± 0.26 | .0100 | 1.92 |
| III + IV ν I | 0.86 ± 0.29 | .0030 | 2.37 |

SUPPLEMENTAL TABLE 2
Estimation of Overall Survival Durations in Patients with NSCLC
per Demographic and Clinicopathological Characteristics Using
Multivariate Cox Proportional Hazards Regression Models

| | Parameter | | Hazard |
|---|---|---|---|
| Characteristic | Estimate ± S.E. | P Value | Ratio |
| Model A: CREB | | | |
| Age | 0.04 ± 0.01 | .0002 | 1.04 |
| CREB | 0.41 ± 0.18 | .0200 | 1.51 |
| Pathological T classification | 0.84 ± 0.28 | .0030 | 2.31 |
| (T2 + T3 + T4 ν T1) | | | |
| Pathological N classification | 0.47 ± 0.24 | .0490 | 1.60 |
| (N1 + N2 + N3 ν N0) | | | |
| Model B: p-CREB | | | |
| Age | 0.04 ± 0.01 | .0005 | 1.04 |
| p-CREB | 0.59 ± 0.23 | .0100 | 1.80 |
| Pathological T classification | 0.69 ± 0.28 | .0100 | 2.00 |
| (T2 + T3 + T4 ν T1) | | | |
| Pathological N classification | 0.47 ± 0.24 | .0500 | 1.59 |
| (N1 + N2 + N3 ν N0) | | | |

### REFERENCES

1. Kamath A V, Chhajed P N: Role of bronchoscopy in early diagnosis of lung cancer. Indian J Chest Dis Allied Sci 48:265-269, 2006
2. Jemal A, Siegel R, Ward E, et al: Cancer statistics, 2006. CA Cancer J Clin 56:106-130, 2006
3. Ionescu A M, Schwarz E M, Vinson C, et al: PTHrP modulates chondrocyte differentiation through AP-1 and CREB signaling. J Biol Chem 276:11639-11647, 2001
4. Riccio A, Ahn S, Davenport C M, et al: Mediation by a CREB family transcription factor of NGF-dependent survival of sympathetic neurons. Science 286:2358-2361, 1999
5. Conkright M D, Montminy M: CREB: the unindicted cancer co-conspirator. Trends Cell Biol 15:457-459, 2005
6. Shankar D B, Sakamoto K M: The role of cyclic-AMP binding protein (CREB) in leukemia cell proliferation and acute leukemias. Leuk Lymphoma 45:265-270, 2004
7. Rudolph D, Tafuri A, Gass P, et al: Impaired fetal T cell development and perinatal lethality in mice lacking the cAMP response element binding protein. Proc Natl Acad Sci USA 95:4481-4486, 1998
8. Long F, Schipani E, Asahara H, et al: The CREB family of activators is required for endochondral bone development. Development 128:541-550, 2001
9. De Cesare D, Fimia G M, Sassone-Corsi P: Signaling routes to CREM and CREB: plasticity in transcriptional activation. Trends Biochem Sci 24:281-285, 1999
10. Herzig S, Long F, Jhala U S, et al: CREB regulates hepatic gluconeogenesis through the coactivator PGC-1. Nature 413:179-183, 2001
11. Koo S H, Flechner L, Qi L, et al: The CREB coactivator TORC2 is a key regulator of fasting glucose metabolism. Nature 437:1109-1111, 2005
12. Mayr B, Montminy M: Transcriptional regulation by the phosphorylation-dependent factor CREB. Nat Rev Mol Cell Biol 2:599-609, 2001
13. Desdouets C, Matesic G, Molina C A, et al: Cell cycle regulation of cyclin A gene expression by the cyclic AMP-responsive transcription factors CREB and CREM. Mol Cell Biol 15:3301-3309, 1995
14. Xiang H, Wang J, Boxer LM: The Role of the cAMP-Response Element in the Bcl-2 Promoter in the Regulation of Endogenous Bcl-2 Expression and Apoptosis in Murine B Cells. Mol Cell Biol, 2006
15. McCauslin C S, Heath V, Colangelo A M, et al: CAAT/enhancer-binding protein delta and cAMP-response element-binding protein mediate inducible expression of the nerve growth factor gene in the central nervous system. J Biol Chem 281:17681-17688, 2006
16. Oike Y, Takakura N, Hata A, et al: Mice homozygous for a truncated form of CREB-binding protein exhibit defects in hematopoiesis and vasculo-angiogenesis. Blood 93:2771-2779, 1999
17. Stylianou E, Saklatvala J: Interleukin-1. Int J Biochem Cell Biol 30:1075-1079, 1998
18. Nyormoi O, Bar-Eli M: Transcriptional regulation of metastasis-related genes in human melanoma. Clin Exp Metastasis 20:251-263, 2003
19. Crans-Vargas H N, Landaw E M, Bhatia S, et al: Expression of cyclic adenosine monophosphate response-element binding protein in acute leukemia. Blood 99:2617-2619, 2002
20. Shankar D B, Cheng J C, Sakamoto K M: Role of cyclic AMP response element binding protein in human leukemias. Cancer 104:1819-1824, 2005
21. Shankar D B, Cheng J C, Kinjo K, et al: The role of CREB as a proto-oncogene in hematopoiesis and in acute myeloid leukemia. Cancer Cell 7:351-362, 2005
22. Sofi M, Young M J, Papamakarios T, et al: Role of CRE-binding protein (CREB) in aromatase expression in breast adipose. Breast Cancer Res Treat 79:399-407, 2003
23. Moorehead R A, Sanchez Ohio, Baldwin R M, et al: Transgenic overexpression of IGF-II induces spontaneous lung tumors: a model for human lung adenocarcinoma. Oncogene 22:853-857, 2003
24. Linnerth N M, Baldwin M, Campbell C, et al: IGF-II induces CREB phosphorylation and cell survival in human lung cancer cells. Oncogene 24:7310-7319, 2005
25. Xie S, Price J E, Luca M, et al: Dominant-negative CREB inhibits tumor growth and metastasis of human melanoma cells. Oncogene 15:2069-2075, 1997
26. Abramovitch R, Tavor E, Jacob-Hirsch J, et al: A pivotal role of cyclic AMP-responsive element binding protein in tumor progression. Cancer Res 64:1338-1346, 2004
27. Beasley M B, Brambilla E, Travis W D: The 2004 World Health Organization classification of lung tumors. Semin Roentgenol 40:90-97, 2005
28. Koo J S, Yoon J H, Gray T, et al: Restoration of the mucous phenotype by retinoic acid in retinoid-deficient human bronchial cell cultures: changes in mucin gene expression. Am J Respir Cell Mol Biol 20:43-52, 1999
29. Koo J S, Jetten A M, Belloni P, et al: Role of retinoid receptors in the regulation of mucin gene expression by retinoic acid in human tracheobronchial epithelial cells. Biochem J 338 (Pt 2):351-357, 1999
30. Aggarwal S, Kim S W, Cheon K, et al: Nonclassical action of retinoic acid on the activation of the cAMP response element-binding protein in normal human bronchial epithelial cells. Mol Biol Cell 17:566-575, 2006
31. Gray T, Koo J S, Nettesheim P: Regulation of mucous differentiation and mucin gene expression in the tracheobronchial epithelium. Toxicology 160:35-46, 2001
32. Gray T, Nettesheim P, Basbaum C, et al: Regulation of mucin gene expression in human tracheobronchial epithelial cells by thyroid hormone. Biochem J 353:727-734, 2001
33. Kolodziej ski P J, Musial A, Koo J S, et al: Ubiquitination of inducible nitric oxide synthase is required for its degradation. Proc Natl Acad Sci USA 99:12315-12320, 2002
34. Ju Z, Kapoor M, Newton K, et al: Global detection of molecular changes reveals concurrent alteration of several biological pathways in nonsmall cell lung cancer cells. Mol Genet Genomics 274:141-154, 2005
35. Schuller H M, Cekanova M: NNK-induced hamster lung adenocarcinomas over-express beta2-adrenergic and EGFR signaling pathways. Lung Cancer 49:35-45, 2005
36. Powell C A, Spira A, Derti A, et al: Gene expression in lung adenocarcinomas of smokers and nonsmokers. Am J Respir Cell Mol Biol 29:157-162, 2003
37. Toh C K, Lim W T: Lung cancer in never-smokers. J Clin Pathol, 2006
38. Toh C K, Gao F, Lim W T, et al: Never-smokers with lung cancer: epidemiologic evidence of a distinct disease entity. J Clin Oncol 24:2245-2251, 2006
39. Tsao A S, Liu D, Lee J J, et al: Smoking affects treatment outcome in patients with advanced nonsmall cell lung cancer. Cancer 106:2428-2436, 2006
40. Clark G M, Zborowski D M, Santabarbara P, et al: Smoking History and Epidermal Growth Factor Receptor Expression as Predictors of Survival Benefit from Erlotinib for Patients with Non-Small-Cell Lung Cancer in the National Cancer Institute of Canada Clinical Trials Group Study BR.21. Clin Lung Cancer 7:389-394, 2006
41. Lim S T, Wong E H, Chuah K L, et al: Gefitinib is more effective in never-smokers with non-small-cell lung cancer: experience among Asian patients. Br J Cancer 93:23-28, 2005
42. Du B, Altorki N K, Kopelovich L, et al: Tobacco smoke stimulates the transcription of amphiregulin in human oral epithelial cells: evidence of a cyclic AMP-responsive element binding protein-dependent mechanism. Cancer Res 65:5982-5988, 2005
43. Tsai S T, Yang K Y, Jin Y T, et al: Amphiregulin as a tumor promoter for oral squamous cell carcinoma: involvement of cyclooxygenase 2. Oral Oncol 42:381-390, 2006
44. Fontanini G, De Laurentiis M, Vignati S, et al: Evaluation of epidermal growth factor-related growth factors and receptors and of neoangiogenesis in completely resected stage I-IIIA non-small-cell lung cancer: amphiregulin and microvessel count are independent prognostic indicators of survival. Clin Cancer Res 4:241-249, 1998
45. Mayo L D, Kessler K M, Pincheira R, et al: Vascular endothelial cell growth factor activates CRE-binding protein by signaling through the KDR receptor tyrosine kinase. Biol Chem 276:25184-25189, 2001
46. Melnikova V O, Mourad-Zeidan A A, Lev D C, et al: Platelet-activating factor mediates MMP-2 expression and activation via phosphorylation of cAMP-response element-binding protein and contributes to melanoma metastasis. J Biol Chem 281:2911-2922, 2006
47. Mountain C F: Revisions in the International System for Staging Lung Cancer. Chest 111:1710-1717, 1997

### Example 2

### Suppression of Active CREB Induces Apoptosis and Inhibition of Growth and Survival of Non-Small Cell Lung Cancer Cell

Abstract

Genes that are regulated by cyclicAMP response element binding (CREB) protein suppress apoptosis, induce cell proliferation, and mediate inflammation, angiogenesis and tumor metastasis. Prior to this work, it was not known whether CREB is involved in lung carcinogenesis. We hypothesized that constitutively active CREB is an important target in the treatment of non-small cell lung cancer (NSCLC) and that its inhibition should block cell proliferation and induce apoptosis in NSCLC cells. We found that the NSCLC cell lines we examined overexpressed constitutively active CREB, two of its upstream kinases, phospho-ribosomal s6-kinase (Rsk), phospho-extracellular signal kinase (Erk1/2), and cell survival genes such as Bcl-2 and Bcl-X_{L}. Ectopic expression of a dominant-repressor CREB (KCREB), mutant CREB (CREB133, Ser133A1a), and small interfering (si) RNA against CREB (siCREB) suppressed the cell proliferation of NSCLC (H1734) cells. Furthermore, treatment of H1734 cells with an inhibitor of CREB signaling pathway, Ro-31-8220, inhibited CREB activation by abrogating phospho-Erks and phospho-Rsks. Consequently, the expression Bcl-2 and Bcl-X_{L}, cell proliferation was suppressed and the cell cycle was arrested in the G2/M phase. Suppression of active CREB by KCREB, CERB133 and treatment of Ro-31-8220 resulted in apoptosis, as indicated by caspase-9, caspase-3 activation, poly (ADP-ribose) polymerase cleavage and terminal transferase dUTP nick end labeling staining in H1734 cells. In summary, suppression of CREB by KCREB, CREB133, siCREB, or Ro-31-8220 induced growth arrest and apoptosis in selected NSCLC cells. Thus, our results indicate that active CREB plays an important role in NSCLC cell growth and survival. Agents that suppress CREB activation could have potential therapeutic value for NSCLC treatment.

Introduction

Non-small cell lung cancer (NSCLC) is an aggressive lung cancer that, despite standard treatment strategies such as surgery, radiotherapy, chemotherapy, or a combination of the three, is associated with a poor survival rate. Thus, there is a great need for better therapies for NSCLC. One tactic that looks promising to improve NSCLC treatment is modulating the expression of transcription factors associated with aggressive tumor growth and decreased patient survival. Research during the past few years has indicated that a transcription factor, the cyclic AMP response element-binding (CREB) protein, regulates the expression of various genes such as Bcl-2, Bcl-X_{L}, cyclooxygenase-2, and tumor necrosis factor alpha, that play an important role in cell survival, inflammation, and proliferation (1-3).

CREB has also been shown to be involved in the carcinogenesis of several types of cancer. For example, pharmacological inhibition of CREB activity has antiproliferative effects on cancer cells by inhibiting aromatase expression and estrogen production in breast adipose tissue (4). In pancreatic cells, CREB binds to specific DNA regulatory elements in the somatostatin gene associated with cellular processes such as islet cell differentiation and tumorigenesis (5). Further, the ectopic expression of dominant-repressor CREB (KCREB) demonstrated that the anti-apoptotic effects occurs through the phosphorylation of CREB in dihydrotestosterone stimulated, androgen-dependent prostate cancer cells, while CREB is constitutively active and has an anti-apoptotic effect in androgen-independent prostrate cancer cells (6). CREB is also overexpressed in bone marrow cells from patients with acute lymphoid or myeloid leukemia, suggesting that CREB is involved in leukemogenesis and that its expression can be a biomarker for leukemia (7). Finally, in human melanoma cells, CREB is a mediator of tumor growth and metastasis. Whereas the expression of KCREB, which targets the activating transcription factor-2 with its peptide fragment, sensitizes melanomas to apoptosis and inhibits tumor growth and metastasis in human melanoma cells (8-12).

Although it is not known whether CREB is directly involved in lung cancer, there are several lines of evidence that suggest that it is. For example, in an animal study demonstrated that the transgenic over expression of insulin-like growth factor-II in murine lung epithelium induced spontaneous lung tumors that displayed morphological characteristics similar to those of human pulmonary adenocarcinoma and that contained high levels of phosphorylated CREB (13). In addition, the increased expression of fibronectin in lung carcinoma was associated with the increased expression of CREB(14). Moreover, NSCLC cells often overexpress tumor necrosis factor-alpha, Bcl-X_{L}, Bcl-2, and interleukin-4 (15-17), which are all regulated by CREB, and their suppression can result in apoptosis. Therefore the aim of our study was to determine whether constitutively active CREB (phosphorylated CREB; pCREB) is an important target in the treatment of NSCLC and whether its inhibition blocks cell proliferation and induces apoptosis in NSCLC cells.

We also sought in this study to identify a pharmacologically safe and effective agent that would block constitutively active CREB and its effect on CREB regulated gene expression. We selected Ro-31-8220, an analog of staurosporine and a Pan protein kinase C (PKC) inhibitor, because PKC is an important signal transducer in the CREB activation pathway. The compound staurosporine was originally isolated from Streptomyces as a potential anti-fungal agent can potently induce apoptosis in mammalian cells through PKC. Hence, we reasoned that Ro-31-8220 would in turn inhibit CREB. However, the mechanism by which Ro-31-8220 causes apoptosis in NSCLC cells is still poorly understood.

In summary, we found that pCREB is overexpressed in several squamous cell carcinoma and adenocarcinoma lung cancer cell lines. The suppression of pCREB by Ro-31-8220, through genetic quenching of CREB activity by KCREB or mutant CREB (CREB 133, Ser133A1a) or by knocking down the expression of CREB by small interfering (si) CREB suppressed cell proliferation, inducted apoptosis, and down-regulated the expression of the gene products regulated by CREB.

Materials and Methods

Cell Culture. We obtained four human NSCLC cell lines, H1734 (human lung adenocarcinoma), H226 (human lung squamous cell carcinoma), A549 (human alveolar lung epithelium cell, poorly differentiated), and H292 (human pulmonary mucoepidermoid adenocarcinoma), from the American Type Culture Collection (Rockville, Md.). Cells were cultured in RPMI containing 10% fetal bovine serum. Normal human tracheobronchial epithelial (NHTBE) cells (Clonetics, San Diego, Calif.), which were used as the control, were cultured by the three-dimensional organotypic air-liquid interface method (18). Ro31-8220 was purchased from Calbiochem (La Jolla, Calif.) and was prepared in DMSO.

Western Blot Analysis. Protein extracts from each of NSCLC cells and the NHTBE cells, were prepared by lysing the cells in SDS lysis buffer (250 mM Tris-Cl, pH 6.5, 2% SDS, 4% β-mercaptoethanol, 0.02% bromophenol blue, 10% glycerol) containing protease and phosphatase inhibitors. Standard SDS-PAGE and Western blotting procedures were used to analyze the cell extracts. Nitrocellulose blots were probed with anti-CREB and anti-phospho-specific pSer-133 CREB (Upstate Biotechnology, Waltham, Mass.); anti-Bcl-2, anti-Bcl-X_{L}, anti-phospho-Erk1/2, and anti-Erk1/2 (Santa Cruz Biotechnology, Santa Cruz, Calif.); and anti-phospho-Rsk and anti-Rsk (Cell Signaling Technology, Cambridge, Mass.). To detect the cleavage products of apoptosis, nitrocellulose blots were probed with anti-poly (ADP-ribose) polymerase (PARP), anti-caspase-9, and anti-caspase-3 antibodies (New England Biolabs, Beverly, Mass.). Anti-actin antibodies were purchased from Sigma-Aldrich (St. Louis, Mo.) and the caspase-3 inhibitor Ac-DEVD-CHO was obtained from Promega (Madison, Wis.).

Electrophoretic Mobility Shift Assay. Isolation of nuclear extracts, sequences, preparation of oligonucleotide probes, electrophoretic mobility shift assay (EMSA) was conducted as previously described (19).

For the super-shift analysis, the nuclear extracts from H1734 cells were incubated with antibodies against CREB, pCREB, an unlabeled (cold) probe or CRE mutant oligonucleotide 5′-AGAGATTGCCTGTGGTCAGAGAGCTAG-3′ (bold face, indicates CRE mutant; Santa Cruz Biotechnology, CA) (100-fold) for 30 min at 37° C. The complex was then analyzed by EMSA. The radioactive bands from the dried gels were visualized and quantitated with Phospho-Imager (Molecular Dynamics, Sunnyvale, Calif.) using ImageQuant software (Amersham Biosciences, Piscataway, N.J.).

Immunocytochemistry for CREB Localization. H1734 cells (1×105 cells/ml) were plated on a glass chamber slide (Falcon; BD Biosciences, San Jose, Calif.) for adherence and treated the next day for 4 h with Ro-31-8220. The chambers were removed, and cells were fixed with cold acetone. After a brief washing in PBS, the slides were blocked with 5% normal goat serum in PBS for 1 h and then incubated for 4 h with rabbit polyclonal anti-pCREB antibody (dilution, 1:100). The slides were washed, incubated with goat anti-rabbit IgG-Alexa 594 (Molecular Probes, Eugene, Oreg.) (dilution 1:100) for 1 h, and counter-stained for nuclei with 4′,6-diamidino-2-phenylindole, dihydrochloride (DAPI; 50 ng/ml) (Molecular Probes, Eugene, Oreg.) for 15 min. The slides were mounted with a mounting medium (Sigma-Aldrich), and confocal images were captured with an Olympus FV500 confocal microscope (NY) by using a Plan Apo 60× oil objective.

Transfection and Cytotoxicity Assays. H1734 cells (10,000/0.05 ml) were plated in triplicate in 24-well plates and transfected them with plasmid DNA (50 ng/well) (BD Biosciences) of wild type CREB (CREBwt), KCREB, and CREB133 by using lipofectamine 2000 (Invitrogen, Carlsbad, Calif.) according to the manufacturer's instructions. For siRNA transfection, siRNA SMARTpool sequences targeting human CREB and the non-specific control pool (Dharmacon RNA Technologies, Lafayette, Colo.) were diluted and stored according to the manufacturer's instructions. The H1734 cells were transfected with a final concentration of 100 nM siRNA SMARTpool or non-specific control pool with the siIMPORTER siRNA transfection reagent (Upstate Biotechnology, Waltham, Mass.) according to the manufacturer's instructions. The transfection efficiencies of the control plasmid were consistently above 80%. At 48 h of expression of CREBwt, KCREB, CREB133, and siCREB the cytotoxicity effect was determined with the MTT dye uptake method, as previously described (20). The optical density at 590 nm was measured using a 96-well multi-scanner autoreader (Micro quant-Bio TEK Instruments, Winooski, Vt.).

The cytotoxic activity of Ro-31-8220 was also determined against the all four NSCLC cells and NHTBE cells. Briefly, the cells (2,000/well) were incubated in triplicate in 96-well plates in the presence or absence of indicated test samples in a final volume of 0.2 ml for different days at 37° C. Afterwards, we performed the MTT assay.

Flow Cytometric Analysis. H1734 cells were first synchronized by serum starvation overnight and then exposed to Ro-31-8220 for different lengths of time in the presence of 5% serum. The cells were washed, trypsinized, and fixed in 70% ethanol for 1 h at −20° C. Finally, cells were washed with PBS, resuspended, and stained in PBS containing 25 μg of propidium iodide and 1 μg/ml RNAase a for 30 min at room temperature. The distribution of cells during the cell cycle was then analyzed with a Facscalibur flow cytometer (Becton Dickinson, Bedford, Mass.).

Soft Agar Colony Assay. Single-cell suspensions of the H1734 and H226 cells were treated with or without different concentrations of Ro-31-8220 and then mixed with agarose to a final concentration of 0.35%. Aliquots of 1.5 ml containing 10⁴ cells and 10% fetal calf serum were plated in triplicate in six-well plates over a base layer of 0.7% agarose and allowed to gel. The number of colonies that were greater than 60 μm was counted after 28 days of incubation.

Apoptosis Assay. H1734 cells (200,000) were plated in chamber slides; the next day, transfected with CREBwt, KCREB, and CREB133 DNA (100 ng/chamber), and with 100 nM siCREB. The terminal transferase dUTP nick end labeling (TUNEL) assay was performed according to the manufacturer's instructions (Promega). Briefly, the cells were fixed after 48 h of transfection in 4% formaldehyde and permeabilized with 0.5% Triton-X-100. The cells were incubated on the slides with terminal deoxynucleotidyl transferase and fluorescein-dUTP. They were then co-stained with DAPI and visualized with an Axioskop 40 fluorescence microscope (Carl Zeiss, Thornwood, N.Y.). Three fields were randomly counted for each sample. The images were captured at a magnification of 400× and stored using Axiovision software (Carl Zeiss), as described in the instructions provided by the manufacturer.

Results

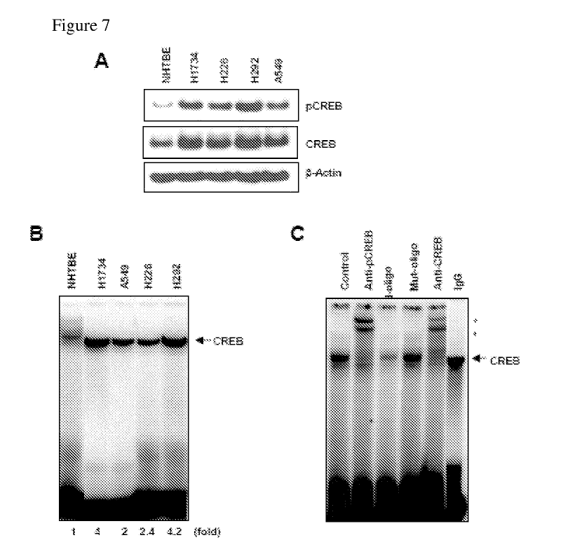

Constitutively Active CREB is overexpressed in NSCLC Cells as Compared with the NHTBE Cells. Western blot analysis showed that constitutively active CREB was over expressed in all four NSCLC cell lines but not in NHTBE cells and CREB was ubiquitously expressed in the NSCLC cells and the NHTBE cells (FIG. 7A). To determine whether active CREB is capable of binding to the CRE-consensus motif 5′-TGACGTCA-3′ we performed an EMSA and a supershift assay. We found that active CREB expressed in all four NSCLC cell lines. The CRE-oligonucleotide consensus binding ability ranging from 2.0 to 4.2 fold (NSCLC) that of the control (NHTBE cells) (FIG. 7B). Next, we determined whether the retarded constitutive band visualized on EMSA was due to CRE-binding specifically to CREB. Nuclear extracts from the H1734 cells were incubated with the ³²P-labeled CRE consensus oligonucleotide and with antibodies to either CREB or pCREB. Both antibodies shifted the band to a higher molecular mass. A 100-fold excess of unlabeled (cold) CRE consensus oligonucleotide caused a substantial reduction of the CRE band, whereas the mutant CRE and non specific IgG did not compete for binding of the CRE binding site (FIG. 7C).

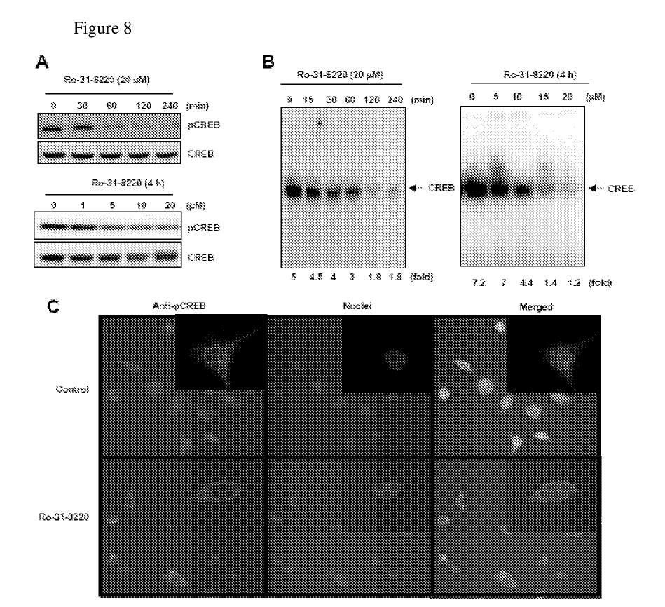

CREB Activity is inhibited by a PKC inhibitor (Ro-31-8220) in H1734 Cells. Incubation of the H1734 cells in a serum free media with Ro-31-8220 substantially suppressed CREB activation in a time dependent and dose dependent manner in the H1743 cells (FIG. 8A). Besides, CRE consensus oligonucleotide binding was suppressed in a time dependent and dose dependent manner (FIG. 8B). Confocal images of cells treated and not treated with Ro-31-8220 for 4 h in a serum free media and then analysed for pCREB showed that Ro-31-8220 induced the nuclear disappearance of pCREB in the H1734 cells (FIG. 8C). These cytological findings were consistent with those from the EMSA results.

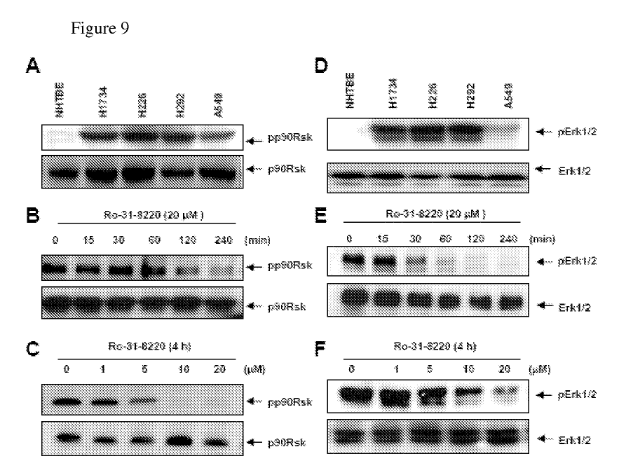

CREB Signaling is Inhibited by a PKC Inhibitor in NSCLC Cells. Rsk is one of the kinases that phosphorylates the CREB transcription factor. Our Western blot analysis showed that all four NSCLC cell lines overexpressed constitutive phospho-Rsk compared with the NHTBE cells (FIG. 9A). Incubation of the H1734 cells in a serum free media with Ro-31-8220 suppressed phospho-Rsk within 2 h (FIG. 9B upper panel); this decrease began at a concentration of 5 μM (FIG. 9B lower panel). Further, the Western blot analysis showed that all four NSCLC cell lines overexpressed constitutive phospho-Erk1/2, which is one of the upstream signaling kinases for Rsk activation and is downstream from the PKC pathway. The NHTBE cells did not express phospho-Erk1/2 (FIG. 9C). Ro-31-8220 treatment in H1734 cells starts decreasing phospho-Erk1/2 expression within 30 min (FIG. 9D upper panel); this decrease began at a concentration of 5 μM (FIG. 9D lower panel).

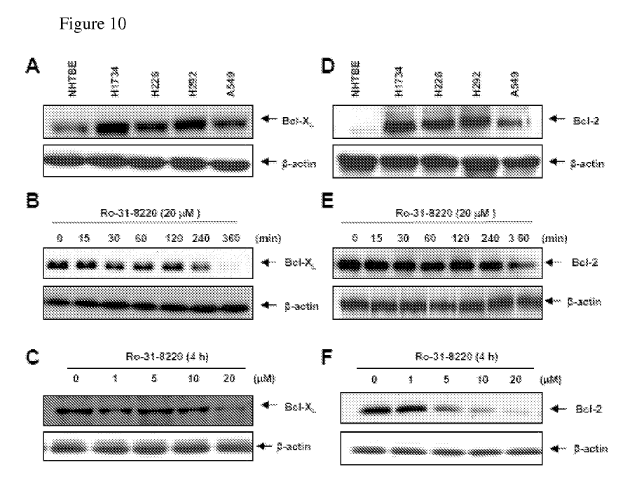

CREB-regulated Anti-apoptotic Genes are down regulated by a PKC inhibitor in NSCLC Cells. Western blot results showed that all four NSCLC cell lines differentially overexpressed Bcl-X_{L} and Bcl-2 compared with the NHTBE cells (FIGS. 10A and 10C). In contrast the expression of Bcl-X_{L} and Bcl-2 was down regulated within 4 h to 6 h of incubation with Ro-31-8220 (FIG. 10B and FIG. 10D upper panel). This result was consistent with the dose response to Ro-31-8220 (FIG. 10B and FIG. 10D lower panel).

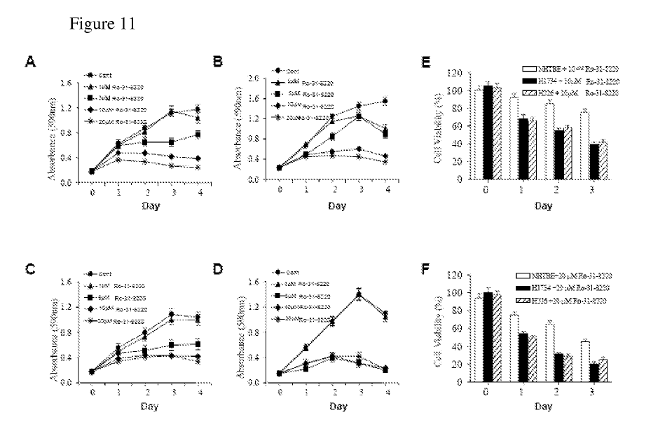

Ro-31-8220 Suppresses Growth of NSCLC Cells. The MTT assay showed that Ro-31-8220 suppressed mitochondrial activity in all four NSCLC cell lines in a dose-dependent manner (FIG. 11A-D). The suppression of mitochondrial activity in the NHTBE cells, however, was significantly less than that in the NSCLC cells (FIG. 11E-F) in a dose dependent manner. Therefore, the results clearly showed that Ro-31-8220 had a more pronounced cytotoxic effect on the NSCLC cells than on the NHTBE cells.

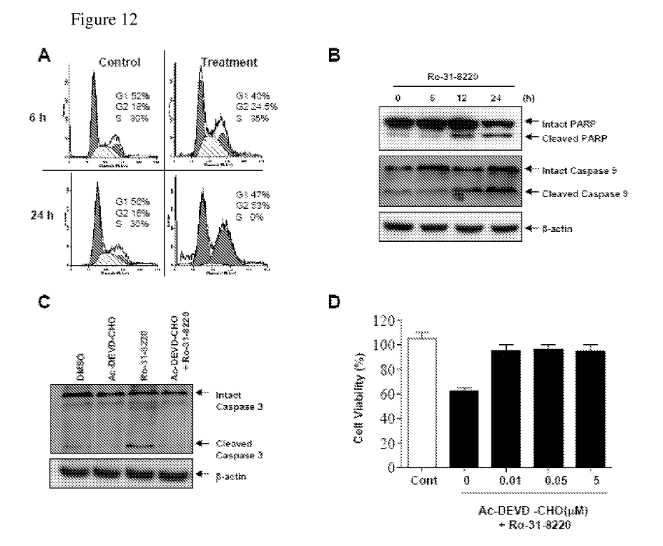

Ro-31-8220 Induces G2/M Cell Cycle Arrest in NSCLC Cells. We examined the effect of Ro-31-8220 on the H1734 cell cycle. After 6 h and 24 h incubation, the percentage of cells in the G2 phase increased from 18% in the controls to 24.5% in the Ro-31-8220-treated cells and the percentage of cells in G2 phase increased from 15% in the controls to 53% in Ro-31-8332 treated cells after 24 h (FIG. 12A), indicating G2/M arrest in a time dependent manner.

Ro-31-8220 Induces Apoptosis in NSCLC Cells. Western blot results clearly showed the cleavage of a 118-kDa PARP protein into an 87-kDa fragment, another hallmark of cells undergoing apoptosis as compare to the untreated cells which did not show any PARP cleavage (FIG. 12B). This was confirmed by our finding of time-dependent activation of caspase-9 (FIG. 12B), as indicated by the cleavage of a 47-kDa to a 37-kDa band. Similarly, the blots showed activation of caspase-3 (FIG. 12C, lane 3), as indicated by cleavage of a 37-kDa band to a 16-kDa band. These findings unequivocally indicate that Ro-31-8220 induced apoptosis in the H1734 cells. To determine whether caspase activation is needed for Ro-31-8220 induced caspase-3 cleavages, H1734 cells were treated with Ro-31-8220 in the presence of the caspase-3 inhibitor Ac-DEVD-CHO and analyzed for caspase-3 cleavage. Ac-DEVD-CHO suppressed the Ro-31-8220 induced caspase-3 cleavage (FIG. 12C, lane 4). To determine whether caspase activation is needed to suppress the cell growth induced by Ro-31-8220, H1734 cells were treated with Ac-DEVD-CHO and examined for Ro-31-8220 induced cytotoxicity. The caspase-3 inhibitor protected the cells from Ro-31-8220 induced cytotoxicity in a dose-dependent manner (FIG. 12D). These results suggest that caspase-3 activation is essential for Ro-31-8220 induced cytotoxicity.

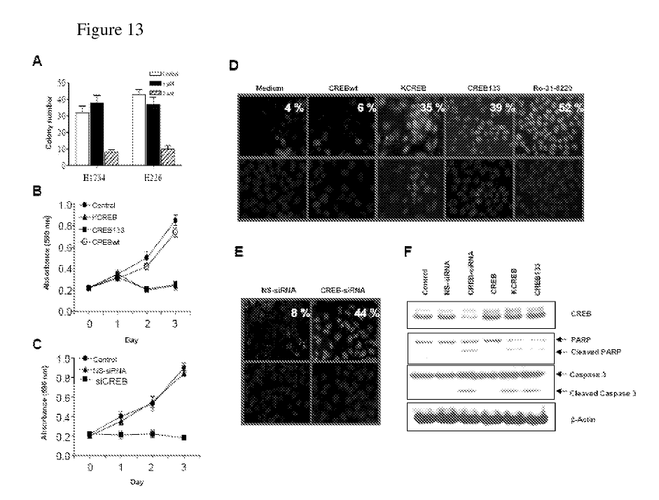

Ro-31-8220 inhibits Colony Formation and Suppression of Constitutively Active CREB Inhibits Cell Proliferation, and Induces Apoptosis in NSCLC Cells. Ro-31-8220 inhibited constitutively active CREB, cell proliferation, induces apoptosis; therefore, we tested whether Ro-31-8220 affects the ability of NSCLC cells to form colonies in soft agar. Treatment of the H1734 and H226 cells with 2 μM Ro-31-8220 resulted in a significant decrease (85%) in colony formation (FIG. 13A). Suppression of constitutively active CREB by the ectopic expression of KCREB, CREB133, and siCREB suppressed the proliferation of the H1734 cells (FIG. 13B and FIG. 13C).

We confirmed apoptosis in the H1734 cells with a TUNEL assay. As shown in FIG. 13D, 35%, 39%, and 44% of the H1734 cells showed apoptosis compared with 6% of control cells (CREBwt) (FIG. 13D) and 8% of non specific siRNA (FIG. 13E) respectively. These data suggest that the depletion of CREB in the H1734 cells made the cells more sensitive to apoptosis. This observation was confirmed by treatment with 5 μM Ro-31-8220 for 48 h; apoptosis was induced in 52% of the cells (FIG. 13D). We performed Western blot analysis to determine whether suppression of CREB by siCREB, KCREB, CREB133 in NSCLC cells would lead to apoptosis. After 48 h of the expression of KCREB, CREB133, and siCREB, the results clearly showed the cleavage of PARP protein and Caspase 3 protein, hallmark of cells undergoing apoptosis as compare to the untreated cells which did not show any PARP and caspase 3 protein cleavages (FIG. 13F).

Discussion

In our study of the role of CREB in the growth and survival of NSCLC cells, we found that CREB is overexpressed and is constitutively active in several human NSCLC lines as determined by Western blot, EMSA, and super-shift analyses. Further, we found that constitutive CREB inhibition directly and/or indirectly in NSCLC inhibits its cell proliferation or induce apoptosis. Therefore we used indirect CREB inhibitor Ro-31-8220 and direct inhibition of CREB by KCREB, CREB 133 and siCREB

Although PKA plays a crucial role in CREB activation but in our studies we checked the ability of a potent PKA inhibitor (H-89) on the cell proliferation of NSCLC cell lines and interestingly we found that PKA inhibitor is not significantly inhibiting the cell growth (supplementary) as compare to pan PKC inhibitor (Ro-31-8220). Therefore we picked up PKC inhibitor for our studies. In our previous studies too, we found that PKA inhibitor doesn't block the retinoic acid activity of CREB while retinoic acid activates CREB via PKC (19) and unequivocally proved that PKC pathway is important in CREB activation. But studies are in progress to explore the role of PKC pathway in related to CREB activation. Further, Ro-31-8220 inhibited NSCLC cell proliferation to a greater extent than it did NHTBE cell proliferation. Also we found that the Ro-31-8220 suppressed constitutive CREB activation in the H1734 cells. Although there are reports that caffeine, glucocorticoid dexamethasone stimulate the proliferation of human lung adenocarcinoma cells, small airway epithelial cells, NCI-H322 cells as well as human immortalized SAECs in a PKA-ERK1/2 and CREB dependent pathway (21, 22). Studies have also shown that CREB is associated with the survival and proliferation of mature B-cells (23). In keeping with this, Ro-31-8220 suppressed the proliferation of U937 human leukemia cells (24), it has also been shown to suppress autocrine growth factors such as interleukin-6 in human cord blood-derived mast cells, mouse bone marrow-derived mast cells, which suppress the proliferation of these cells and MC/9 mouse mast cells (25). Ro-31-8220 has also been shown to suppress cell survival nuclear transcription factor kappa B-dependent transcription in pulmonary A549 cells (26). CREB is associated with NSCLC cell survival as demonstrated by constitutive overexpression of CREB regulated cell survival proteins such as Bcl-2 and Bcl-X_{L} in NSCLC cells and the expression of the anti-apoptotic proteins such as Bcl-2 and Bcl-X_{L} was down regulated as a result of the suppression of active CREB by Ro-31-8220. Our findings agree with other reported studies that Bcl-2 and Bcl-X_{L} are regulated by CREB (14, 27, 28). We also found that upstream kinases such as phospho-Rsk and phospho-Erk1/2, which are involved in the CREB signaling pathway, are over expressed and constitutively active in several human NSCLC lines. In particular, Ro-31-8220 inhibited the constitutive phosphorylation of Erk1/2, which led to the inhibition of phospho-Rsk. Our results agreed with previously reported findings that phospho-Rsk and phospho-Erk1/2 are involved in the CREB activation pathway (29, 30) and our own studies showed that PKC is involved in CREB signaling pathway (19) but still studies are in progress to explore how the CREB signaling candidates including PKC are connected and play role in NSCLC cell survival.

This raises question of how indirectly blocking active CREB by Ro-31-8220 inhibits cell growth and induces apoptosis in NSCLC cells. Cell proliferation requires a multitude of events to translate a mitogenic signal into cell division. Mitotic division is a key event in cell proliferation, whereby cytokinetic fission which follows the segregation of the condensed chromatin, results in a doubling of cell numbers (31). Interestingly, microtubule drugs that arrests cell division such as taxanes have proven effective in the treatment of cancers (32). Ro-31-8220-treated cells like wise did not undergo mitotic cell division because the G2 phase was blocked, resulting in apoptosis. Apoptosis was accompanied by the activation of caspase-9 and caspase-3 and by PARP cleavage. The finding that Ac-DEVD-CHO inhibited this induction of apoptosis and proliferation support a direct link with caspase activation. Our results agree with the studies which showed that Ro-31-8220 induces apoptosis in U937 human leukemia cells (24), and CREB's repression pathway in glioblastoma U87MG cells involves a PKC-mediated pathway (33). At very low concentrations Ro-31-8220 decreases the ability of NSCLC cells to form colonies in agar, suggesting that CREB and its signaling pathway are involved in the tumorigenicity and metastatic potential of NSCLC. There is report that overexpression of CREBwt in the normal bronchial epithelial cell line (HBE135) enchances soft agar growth (34) suggesting that CREB and its associated proteins plays a significant role in lung adenocarcinoma. RNA interference has been used to inhibit cell proliferation and to induce apoptosis in breast cancer cells by knocking down the expression of surviving (35). Studies have also found that retroviral based approaches for delivering siRNA into tissue-cultured mammalian cells are powerful (36, 37). These studies have marked the beginning of a new era in the genetic manipulation of human cancer development by allowing cell survival genes to be downregulated by RNA interference. To the best of our knowledge, this study is the first to show that the knockdown of CREB by siCREB leads to anti-proliferation and apoptosis in NSCLC cells. Our results unequivocally prove that CREB plays an important role in NSCLC cell survival; the knockdown of CREB expression leads to cell death. Other studies have shown that the expression of KCREB decreases the tumorigenicity and metastatic potential in nude mice (10, 11, 38), reduces the resistance of human melanoma cells to radiation (8), and reduces adipogenesis (39). It is clear from our studies that suppressing active CREB indirectly by chemical drugs such as Ro-31-8220, directly knocking down of the expression of CREB by siCREB, blocking phosphorylation of CREB by CREB133, and forming an inactive dimer with CREB by KCREB suppresses cell proliferation and induces apoptosis in NSCLC. We believe that further study of the agents that inhibit active CREB or knocking down CREB expression is needed to improve the treatment of NSCLC.

### REFERENCES

1. Yeo, M. G., Yoo, Y. G., Choi, H. S., Pak, Y. K., and Lee, M. O. Negative Cross-talk between Nur77 and SHP and Its Role in Apoptotic Cell Death of Hepatoma Cells. Mol Endocrinol, 2004.
2. Garcia-Silva, S, and Aranda, A. The thyroid hormone receptor is a suppressor of ras-mediated transcription, proliferation, and transformation. Mol Cell Biol, 24: 7514-7523, 2004.
3. Kundu, J. K., Mossanda, K. S., Na, H. K., and Surh, Y. J. Inhibitory effects of the extracts of Sutherlandia frutescens (L.) R. Br. and Harpagophytum procumbens DC. on phorbol ester-induced COX-2 expression in mouse skin: AP-1 and CREB as potential upstream targets. Cancer Lett, 218: 21-31, 2005.
4. Sofi, M., Young, M. J., Papamakarios, T., Simpson, E. R., and Clyne, C. D. Role of CRE-binding protein (CREB) in aromatase expression in breast adipose. Breast Cancer Res Treat, 79: 399-407, 2003.
5. Habener, J. F., Vallejo, M., and Hoeffler, J. P. Factors that determine cell-specific gene expression in pancreatic endocrine tumor cells. Horm Res, 32: 61-66, 1989.
6. Unni, E., Sun, S., Nan, B., McPhaul, M. J., Cheskis, B., Mancini, M. A., and Marcelli, M. Changes in androgen receptor nongenotropic signaling correlate with transition of LNCaP cells to androgen independence. Cancer Res, 64: 7156-7168, 2004.
7. Shankar, D. B., Cheng, J. C., Kinjo, K., Federman, N., Moore, T. B., Gill, A., Rao, N. P., Landaw, E. M., and Sakamoto, K. M. The role of CREB as a proto-oncogene in hematopoiesis and in acute myeloid leukemia. Cancer Cell, 7: 351-362, 2005.
8. Yang, Y. M., Dolan, L. R., and Ronai, Z. Expression of dominant negative CREB reduces resistance to radiation of human melanoma cells. Oncogene, 12: 2223-2233, 1996.
9. Xie, S., Price, J. E., Luca, M., Jean, D., Ronai, Z., and Bar-Eli, M. Dominant-negative CREB inhibits tumor growth and metastasis of human melanoma cells. Oncogene, 15: 2069-2075, 1997.
10. Jean, D., Harbison, M., McConkey, D. J., Ronai, Z., and Bar-Eli, M. CREB and its associated proteins act as survival factors for human melanoma cells. J Biol Chem, 273: 24884-24890, 1998.
11. Jean, D. and Bar-Eli, M. Regulation of tumor growth and metastasis of human melanoma by the CREB transcription factor family. Mol Cell Biochem, 212: 19-28, 2000.
12. Bhoumik, A., Gangi, L., and Ronai, Z. Inhibition of melanoma growth and metastasis by ATF2-derived peptides. Cancer Res, 64: 8222-8230, 2004.
13. Moorehead, R. A., Sanchez, O. H., Baldwin, R. M., and Khokha, R. Transgenic overexpression of IGF-II induces spontaneous lung tumors: a model for human lung adenocarcinoma. Oncogene, 22: 853-857, 2003.
14. Abou El Hassan, M. A., Mastenbroek, D.C., Gerritsen, W. R., Giaccone, G., and Kruyt, F. A. Overexpression of Bcl2 abrogates chemo- and radiotherapy-induced sensitisation of NCI-H460 non-small-cell lung cancer cells to adenovirus-mediated expression of full-length TRAIL. Br J Cancer, 91: 171-177, 2004.
15. Liu, X., Yue, P., Zhou, Z., Khuri, F. R., and Sun, S. Y. Death receptor regulation and celecoxib-induced apoptosis in human lung cancer cells. J Natl Cancer Inst, 96: 1769-1780, 2004.
16. Nguyen, D. M., Chen, G. A., Reddy, R., Tsai, W., Schrump, W. D., Cole, G., Jr., and Schrump, D. S. Potentiation of paclitaxel cytotoxicity in lung and esophageal cancer cells by pharmacologic inhibition of the phosphoinositide 3-kinase/protein kinase B (Akt)-mediated signaling pathway. J Thorac Cardiovasc Surg, 127: 365-375, 2004.
17. Shankaranarayanan, P. and Nigam, S. IL-4 induces apoptosis in A549 lung adenocarcinoma cells: evidence for the pivotal role of 15-hydroxyeicosatetraenoic acid binding to activated peroxisome proliferator-activated receptor gamma transcription factor. J Immunol, 170: 887-894, 2003.
18. Gray, T. E., Guzman, K., Davis, C. W., Abdullah, L. H., and Nettesheim, P. Mucociliary differentiation of serially passaged normal human tracheobronchial epithelial cells. Am J Respir Cell Mol Biol, 14: 104-112, 1996.
19. Aggarwal, S., Kim, S. W., Cheon, K., Tabassam, F. H., Yoon, J. H., and Koo, J. S. Nonclassical Action of Retinoic Acid on the Activation of the cAMP Response Element-binding Protein in Normal Human Bronchial Epithelial Cells. Mol Biol Cell, 2005.
20. Manna, S. K., Zhang, H. J., Yan, T., Oberley, L. W., and Aggarwal, B. B. Overexpression of manganese superoxide dismutase suppresses tumor necrosis factor-induced apoptosis and activation of nuclear transcription factor-kappaB and activated protein-1. J Biol Chem, 273: 13245-13254, 1998.
21. Al-Wadei, H. A., Takahasi, T., and Schuller, H. M. PKA-dependent growth stimulation of cells derived from human pulmonary adenocarcinoma and small airway epithelium by dexamethasone. Eur J Cancer, 41: 2745-2753, 2005.
22. Al-Wadei, H. A., Takahashi, T., and Schuller, H. M. Caffeine stimulates the proliferation of human lung adenocarcinoma cells and small airway epithelial cells via activation of PKA, CREB and ERK1/2. Oncol Rep, 15: 431-435, 2006.
23. Blois, J. T., Mataraza, J. M., Mecklenbrauker, I., Tarakhovsky, A., and Chiles, T. C. B cell receptor-induced cAMP-response element-binding protein activation in B lymphocytes requires novel protein kinase Cdelta. J Biol Chem, 279: 30123-30132, 2004.
24. Choi, Y. J., Park, J. W., Woo, J. H., Kim, Y. H., Lee, S. H., Lee, J. M., and Kwon, T. K. Failure to activate caspase 3 in phorbol ester-resistant leukemia cells is associated with resistance to apoptotic cell death. Cancer Lett, 182: 183-191, 2002.
25. Boudreau, R. T., Garduno, R., and Lin, T. J. Protein phosphatase 2A and protein kinase Calpha are physically associated and are involved in Pseudomonas aeruginosa-induced interleukin 6 production by mast cells. J Biol Chem, 277: 5322-5329, 2002.
26. Catley, M. C., Cambridge, L. M., Nasuhara, Y., Ito, K., Chivers, J. E., Beaton, A., Holden, N. S., Bergmann, M. W., Barnes, P. J., and Newton, R. Inhibitors of protein kinase C (PKC) prevent activated transcription: role of events downstream of NF-kappaB DNA binding. J Biol Chem, 279: 18457-18466, 2004.
27. Daniel, J. C. and Smythe, W. R. The role of Bcl-2 family members in non-small cell lung cancer. Semin Thorac Cardiovasc Surg, 16: 19-27, 2004.
28. Karczmarek-Borowska, B., Filip, A., Zdunek, M., Korobowicz, E., Wojcierowski, J., Korszen-Pilecka, I., and Furmanik, F. The preliminary estimation of bcl-2, bcl-X(L) and p53 genes expression in locally advanced non-small cell lung cancer. Ann Univ Mariae Curie Sklodowska [Med], 58: 124-131, 2003.
29. Xing, J., Ginty, D. D., and Greenberg, M. E. Coupling of the RAS-MAPK pathway to gene activation by RSK2, a growth factor-regulated CREB kinase. Science, 273: 959-963, 1996.
30. Impey, S., Obrietan, K., Wong, S. T., Poser, S., Yano, S., Wayman, G., Deloulme, J. C., Chan, G., and Storm, D. R. Cross talk between ERK and PKA is required for Ca2+ stimulation of CREB-dependent transcription and ERK nuclear translocation. Neuron, 21: 869-883, 1998.
31. Casenghi, M., Meraldi, P., Weinhart, U., Duncan, P. I., Korner, R., and Nigg, E. A. Polo-like kinase 1 regulates Nlp, a centrosome protein involved in microtubule nucleation. Dev Cell, 5: 113-125, 2003.
32. Wood, K. W., Cornwell, W. D., and Jackson, J. R. Past and future of the mitotic spindle as an oncology target. Curr Opin Pharmacol, 1: 370-377, 2001.
33. Perry, C., Sklan, E. H., and Soreq, H. CREB regulates AChE-R-induced proliferation of human glioblastoma cells. Neoplasia, 6: 279-286, 2004.
34. Linnerth, N. M., Baldwin, M., Campbell, C., Brown, M., McGowan, H., and Moorehead, R. A. IGF-II induces CREB phosphorylation and cell survival in human lung cancer cells. Oncogene, 24: 7310-7319, 2005.
35. Xiong, H., Yu, S., Hu, G., and Zhuang, L. Effects of Survivin expression suppressed by short hairpin RNA on MCF-7 cells. J Huazhong Univ Sci Technolog Med Sci, 26: 305-307, 2006.
36. Yang, G., Thompson, J. A., Fang, B., and Liu, J. Silencing of H-ras gene expression by retrovirus-mediated siRNA decreases transformation efficiency and tumorgrowth in a model of human ovarian cancer. Oncogene, 22: 5694-5701, 2003.
37. Brummelkamp, T. R., Bernards, R., and Agami, R. Stable suppression of tumorigenicity by virus-mediated RNA interference. Cancer Cell, 2: 243-247, 2002.
38. Jean, D. and Bar-Eli, M. Targeting the ATF-1/CREB transcription factors by single chain Fv fragment in human melanoma: potential modality for cancer therapy. Crit Rev Immunol, 21: 275-286, 2001.
39. Reusch, J. E., Colton, L. A., and Klemm, D. J. CREB activation induces adipogenesis in 3T3-L1 cells. Mol Cell Biol, 20: 1008-1020, 2000.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating a cancer characterized by overexpression of cyclic adenosine monophosphate response element-binding protein (CREB) or phosphorylated CREB (p-CREB), comprising:
administering to a patient suffering from the cancer a pharmaceutically-effective amount of a composition comprising a compound selected from the group consisting of 2-{1-[3-(Amidinothio)propyl]-1H-indol-3-yl}-3-(1-methylindol-3-yl)maleimide, naphthol analogs having the structure I: 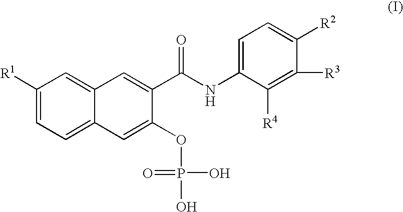
wherein R¹ is selected from the group consisting of —H, —F, —Cl, —Br, and —I;
R² is selected from the group consisting of —H, —F, —Cl, —Br, —I, —C₁₋₆ alkyl, —C₁₋₆ alkenyl, and —C₁₋₆ alkynyl;
R³ is selected from the group consisting of —H and —NO₂; and
R⁴ is selected from the group consisting of —H, —C₁₋₆ alkyl, —C₁₋₆ alkenyl, —C₁₋₆ alkynyl, —OC₁₋₆ alkyl, —OC₁₋₆ alkenyl, and —OC₁₋₆ alkynyl;
and salts and esters thereof.

2. The method of claim 1, wherein the naphthol analog is selected from the group consisting of the compound having structure I wherein R¹ is —H, R² is —Cl, R³ is —H, and R⁴ is —H (naphthol AS-E phosphate); the compound having structure I wherein R¹ is —Br, R² is —H, R³ is —H, and R⁴ is —OCH₃ (naphthol AS-BI phosphate); the compound having structure I wherein R¹ is —H, R² is —H, R³ is —NO₂, and R⁴ is —H (naphthol AS-BS phosphate); the compound having structure I wherein R¹ is —H, R² is —CH₃, R³ is —H, and R⁴ is —CH₃ (naphthol AS-MX phosphate); and the compound having structure I wherein R¹ is —H, R² is —Cl, R³ is —H, and R⁴ is —CH₃ (naphthol AS-TR phosphate).

3. The method of claim 2, wherein the naphthol analog is naphthol AS-E phosphate.

4. The method of claim 1, wherein the cancer is selected from the group consisting of non-small cell lung cancer (NSCLC), adenocarcinoma, head and neck cancer, breast cancer, liver cancer, colon cancer, brain cancer, leukemia, larynx cancer, tonsil cancer, thyroid gland cancer, kidney cancer, cervical cancer, uterine cancer, ovarian cancer, testicular cancer, prostate cancer, and gall bladder cancer.

5. The method of claim 1, wherein the composition further comprises a pharmaceutically-acceptable carrier.

6. The method of claim 1, wherein administering comprises a dosage from about 0.001 mg naphthol analog/kg body weight per day to about 1 g naphthol analog/kg body weight per day.

7. A composition, comprising:
a compound selected from the group consisting of 2-{1-[3-(Amidinothio)propyl]-1H-indol-3-yl}-3-(1-methylindol-3-yl)maleimide, naphthol analogs having the structure I: 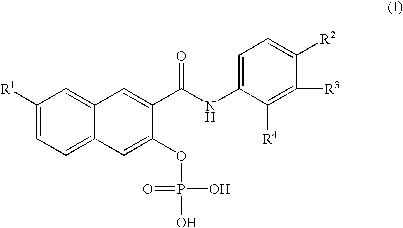
wherein R¹ is selected from the group consisting of —H, —F, —Cl, —Br, and —I;
R² is selected from the group consisting of —H, —F, —Cl, —Br, —I, —C₁₋₆ alkyl, —C₁₋₆ alkenyl, and —C₁₋₆ alkynyl;
R³ is selected from the group consisting of —H and —NO₂; and
R⁴ is selected from the group consisting of —H, —C₁₋₆ alkyl, —C₁₋₆ alkenyl, —C₁₋₆ alkynyl, —OC₁₋₆ alkyl, —OC₁₋₆ alkenyl, and —OC₁₋₆ alkynyl;
and salts and esters thereof; and,
a pharmaceutically-acceptable carrier.
